(19) United States
(12) Patent Application Publication
(10) Pub. No.: US 2007/0110210 A1
(43) Pub. Date: May 17, 2007

(54) X-RAY CT APPARATUS AND X-RAY CT FLUOROSCOPIC APPARATUS

(76) Inventors: Akihiko Nishide, Tokyo (JP); Naoyuki Kawachi, Tokyo (JP); Akira Izuhara, Tokyo (JP); Makoto Gohno, Tokyo (JP); Motoki Watanabe, Tokyo (JP)

Correspondence Address: Patrick W. Rasche;Armstrong Teasdale LLP, Suite 2600, One Metropolitan Square, St. Louis, MO, 63102, US

(21) Appl. No.: 11/559,494

(22) Filed: Nov. 14, 2006

(30) Foreign Application Priority Data
Nov. 15, 2005 (JP) 2005-329714

(51) Int. Cl. H05G 1/60; A61B 6/00; G01N 23/00; G21K 1/12

(52) U.S. Cl. 378/4

(57) ABSTRACT

Tomography or X-ray CT fluoroscopy reduced in exposure to radiation in an X-ray CT apparatus or an X-ray CT fluoroscopic apparatus is to be substantialized. A channel-direction X-ray collimator or a beam forming X-ray filter is positionally controlled in the channel direction to carry out X-ray data acquisition while irradiation with X-rays limited to only the region of interest. Either the profile area of the whole subject is obtained or the profile area of the whole subject is predicted from views or scout images of irradiation of the whole subject out of X-ray projection data. Image reconstruction of views not irradiating the whole subject out of the collected X-ray projection data is carried out by predicting lacking parts from the profile area of the whole subject and making corrections accordingly. It is thereby made possible to irradiate only the region of interest with X-rays to reduce the exposure of the subject of tomography by the X-ray CT apparatus to X-rays or the exposure the subject to X-rays and the exposure of the operator's hands to radiation at the time of puncturing in X-ray CT fluoroscopy.

### CROSS REFERENCE TO RELATED APPLICATIONS

This application claims the benefit of Japanese Application No. 2005-329714 filed Nov. 15, 2005.

### BACKGROUND OF THE INVENTION

The present invention relates to an X-ray CT (Computed Tomography) imaging method and an X-ray CT apparatus, and relates to an X-ray CT image reconstructing method and an X-ray CT apparatus for projection data of which part of the channel is lacking or projection data including substances which are hard to transmit X-ray (such as metals). It relates to an X-ray CT image reconstructing method and an X-ray CT apparatus for projection data to be acquired by a collimator in the channel direction, enabled to realize low exposure to radiation.

It relates to an X-ray CT fluoroscopic image reconstructing method and an X-ray CT fluoroscopic apparatus by which the exposure of the operator's hands to X-rays is reduced.

Demands are rising for reductions in the dose of radiation to which patients are exposed in X-ray CT. In order to realize low exposure, realization of a significant reduction in radiation exposure is sought by building up low exposure techniques even if each of the exposure reducing effects is only modest. Demands are also rising for reductions in the exposure of the operator's hands to radiation at the time of puncturing in X-ray CT fluoroscopy.

The present invention relates to a technique of attempting image reconstruction while appropriately predicting the profile lacking in the channel direction and supplementing the pertinent projection data by using “information on every profile area in the reconstructed field of view”, which is one of the characteristic parameters obtained from a scout image or X-ray projection data of a view not lacking in X-ray projection data in the channel direction to add the part insufficient in X-rays lacking in some channels by irradiating only the region of interest with X-rays by using a channel-direction X-ray collimator or a beam-forming X-ray filter though this is inconsistent with the principle of image reconstruction “to achieve image reconstruction by irradiating only a part with X-rays instead of irradiating the whole object area present in the field of view of reconstruction with X-rays”.

It relates to a technique of appropriately performing image reconstruction by supplementing deteriorated X-ray projection data by using a similar technique even where the S/N ratio is extremely poor on some channels of X-ray projection data.

A challenge to the present invention consists in whether or not image reconstruction can be appropriately achieved by performing positional control in the channel direction or aperture width control of such a collimator or a beam-forming X-ray filter as irradiating with X-rays only the minimum area of the region of interest of the subject.

Conventionally, where X-ray projection data lacked projection data in the channel direction or contained substances which could hardly transmit X-rays (such as metals) and were poor in S/N ratio, inconsistency occurred in the X-ray projection data of the tomogram because the whole section of the subject could not be included in the imaged area or because X-ray projection data corresponding to the section of the subject could not be obtained. For this reason, other regions of the subject than the interest region were also irradiated with X-rays and the whole section of the subject was included in the imaged area. As a result, it was difficult to reduce radiation exposure in such a way that only the region of interest was irradiated with X-rays. Moreover, there was no channel-direction collimator which could move in such a channel direction that only the region of interest was irradiated with X-rays. Nor was known a method by which X-ray irradiation was focused on the region of interest with a beam forming X-ray filter and the surrounding areas were hardly irradiated with X-rays.

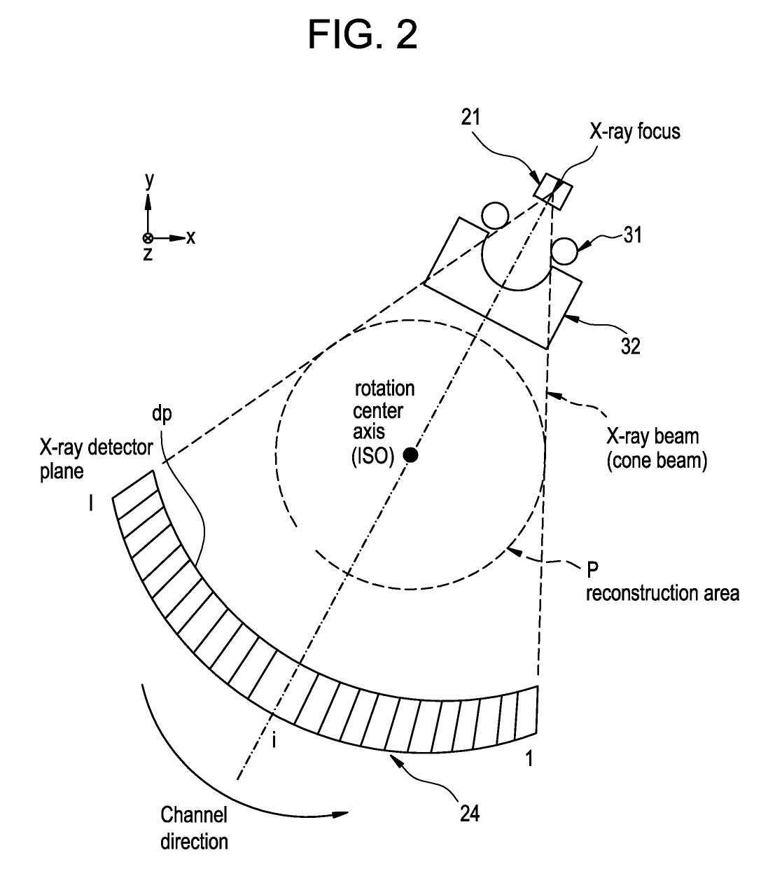

Conventionally, it was usual for X-ray CT apparatuses to obtain tomograms in the image reconstruction area by irradiating all the channels of X-ray detectors as shown in FIG. 2. The following reference contains an example of usual X-ray tomography (see JP-A No. 152925/2000 for instance).

The present invention relates to an X-ray CT apparatus using a multi-row X-ray detector, which so effects control that an appropriate position in the z direction is irradiated by having an collimator perform tracking in the z-direction (the direction of slice thickness), which is the advancing direction of an image pickup table.

In this case, however, even where the region desired to be picked up was only a part of the tomographic field of view, which is an xy plane, the whole area of the subject was irradiated with X-rays. For instance, even where only one of the lungs or the heart was desired to be tomographed, both lungs plus the heart were irradiated with X-rays.

### SUMMARY OF THE INVENTION

In view of this, an object of the present invention is to realize an X-ray CT apparatus which performs image reconstruction, even where projection data have become lacking in the channel direction, by correcting the projection data to provide a tomogram of higher picture quality.

Another object is to realize an X-ray CT apparatus which is equipped with at least either one of a channel-direction X-ray collimator and a beam forming X-ray filter which irradiates with X-rays only the region of interest of the region to be tomographed, tracks the region of interest of the region to be tomographed and performs tomography without irradiating the unnecessary area with X-rays or with reduced irradiation, and correcting on the basis of prediction from a scout image or characteristic parameters, of which one example is the profile area of projection data not lacking in X-ray projection data in the channel direction or not deteriorated in S/N ratio, X-ray projection data in any lacking part or deteriorated in S/N ratio to make possible imaging with reduced exposure to radiation.

Still another object is to realize an X-ray CT fluoroscopic apparatus which limits the X-ray irradiated area with the channel-direction X-ray collimator or beam forming X-ray filter to reduce the exposure of the operator, especially the exposure of the operator's hands, to radiation at the time of puncturing in X-ray CT fluoroscopy.

Therefore according to the invention, in order to so control the channel-direction X-ray collimator as to irradiate only the region to be imaged with X-rays, only the region of interest may be caused to be irradiated with X-rays by subjecting the position and aperture width of the X-rays of the channel-direction X-ray collimator to feedback control while monitoring the output of an X-ray detector or the position of the region desired to be imaged, which is known in advance, may be calculated with respect to each view position and only the region of interest may be caused to be irradiated with X-rays by subjecting the position and aperture width of the X-rays of the channel-direction X-ray collimator to forward control. The X-ray projection data obtained then lack in part of projection data because the whole of the tomogram screen where the subject is present is not subjected to fluoroscopy. For this reason, in order to improve the picture quality of the tomogram of the region of interest of the region to be imaged, it is necessary to predict the X-ray projection data by using characteristic parameters, of which one example is the profile area of the part of the lacking projection data and, after performing addition and correction, to reconstruct the image.

For this prediction of projection data, a profile area corresponding to the whole imaging field of view in the z coordinate position where the subject is present is figured out in advance from the z coordinate of each position where a tomogram is desired in performing scout scanning and the scout image profile of the imaging position. The difference between this profile area of the whole imaging field of view and the X-ray projection data collimator-controlled in the channel direction is also figured out in advance. This difference corresponds to the part not imaged in the projection data of the area limited by the channel-direction X-ray collimator, and an equivalent of this is correctively added to the projection data which are collimator-controlled in the channel direction. By reconstructing an image from the corrected projection data, a tomogram of normal picture quality can be obtained by preventing the artifact and partial or total CT value rise or fall of the tomogram in the region desired to be imaged.

Also, where only the region of interest is much irradiated with X-rays and other areas are little irradiated with X-rays by using a beam forming X-ray filter (also known as a wedge filter, an add-on filter or a bow tie filter) instead of the channel-direction X-ray collimator, similar correction can be accomplished to give an appropriate tomogram.

Also by applying the above-described imaging method and image reconstruction method to an X-ray CT fluoroscopic apparatus, not only the exposure of the subject to radiation but also the dose of exposure of the operator's hands to X-rays at the time of puncturing can be reduced. In this case, setting can be so made as not let the operator's hands enter the region of interest of irradiation with X-rays.

In its first aspect, the present invention provides an X-ray CT apparatus comprising X-ray data acquisition means which, while rotating an X-ray generating device and a multi-row X-ray detector which detects X-rays in an opposing manner, collects X-ray projection data transmitted by a subject positioned in-between; image reconstructing means which performs image reconstruction from the projection data collected from that X-ray data acquisition means; image display means which displays a tomogram having undergone image reconstruction; and imaging condition setting means which sets various imaging conditions of tomography, the X-ray CT apparatus being characterized in that it has such image reconstructing means as performs image reconstruction by correcting X-ray projection data lacking in some channels or deteriorated in S/N ratio.

In the X-ray CT apparatus in the first aspect, when the subject is fully contained in the imaging field of view of the X-ray CT apparatus, the total profile area is constant in the case of a normal parallel beam.

Also in the case of a fan beam, it can be considered approximately constant.

By utilizing such characteristics of the X-ray CT apparatus, even where some channels are lacking or the S/N ratio is deteriorated, image reconstruction can be accomplished after making corrections by adding X-ray projection data at the time of image reconstruction.

In its second aspect, the invention provides an X-ray CT apparatus characterized in that it has, in the X-ray CT apparatus of the first aspect, image reconstructing means which, when X-ray projection data lacking in some channels or deteriorated in S/N ratio are to be corrected, uses projection data of views not lacking in X-ray projection data.

In the X-ray CT apparatus in the second aspect, in addition to the first aspect, where the subject is not circular but is oval-shaped or can be approximated to an oval shape, projection data can be collected free from lacking in the channel direction or deterioration in S/N ratio in some view directions if the aperture width of the X-ray beam in the channel direction is sufficient to some extent. By using such X-ray projection data, even where some channels are lacking or the S/N ratio is deteriorated, image reconstruction can be accomplished after making corrections by adding X-ray projection data at the time of image reconstruction.

In its third aspect, the invention provides an X-ray CT apparatus characterized in that it has, in the X-ray CT apparatus of either the first or the second aspect, image reconstructing means which, when X-ray projection data lacking in some channels or deteriorated in S/N ratio are to be corrected, uses characteristic parameters of views not lacking in X-ray projection data.

In the X-ray CT apparatus in the third aspect, in addition to either the first or second aspect, where the subject is not circular but is oval-shaped or can be approximated to an oval shape, characteristic parameters such as the profile area of the X-ray projection data obtained where X-ray projection data can be collected free from lacking in the channel direction or deterioration in S/N ratio in some view directions if the aperture width of the X-ray beam in the channel direction is sufficient to some extent. By using such characteristic parameters, even where some channels are lacking or the S/N ratio is poor, image reconstruction can be accomplished after making corrections by adding X-ray projection data at the time of image reconstruction.

In its fourth aspect, the invention provides an X-ray CT apparatus characterized in that it has, in the X-ray CT apparatus of the first aspect, image reconstructing means which, when X-ray projection data lacking in some channels or deteriorated in S/N ratio are to be corrected, uses scout images.

In its fourth aspect, the invention provides an X-ray CT apparatus characterized in that it can, in addition to the X-ray CT apparatus of the first aspect, obtain the total profile area of the subject by using scout images of the subject. Usually, scout images are collected from at least one direction or two directions out of the 0-degree direction and the 90-degree direction. Since the arrangement in scout imaging is usually such that the whole subject can be imaged, the total profile area of the subject can be known. By using such scout images, even where some channels are lacking or the S/N ratio is poor, image reconstruction can be accomplished after making corrections by adding X-ray projection data at the time of image reconstruction.

In its fifth aspect, the invention provides an X-ray CT apparatus characterized in that it has, in the X-ray CT apparatus of either the first or the fourth aspect, image reconstructing means which, when X-ray projection data lacking in some channels or deteriorated in S/N ratio are to be corrected, uses characteristic parameters of scout images.

In its fifth aspect, the invention provides an X-ray CT apparatus characterized in that it can, in addition to the first aspect and the fourth aspect, it can obtain X-ray projection data in the z-directional position in which the subject is desired to be imaged if scout images of the subject in at least one direction out of the 0-degree direction and the 90-degree direction or any other direction are collected, and characteristic parameters such as the profile area of those X-ray projection data can be figured out. By using these characteristic parameters, even where some channels are lacking or the S/N ratio is poor, image reconstruction can be accomplished after making corrections by adding X-ray projection data at the time of image reconstruction.

In its sixth aspect, the invention provides an X-ray CT apparatus characterized in that, in the X-ray CT apparatus of either the third or the fifth aspect, it has image reconstructing means in which the characteristic parameters include a profile area.

In the X-ray CT apparatus in the sixth aspect, X-ray projection data of the subject in the z-directional position in which the subject is desired to be imaged can be obtained from scout images in at least one direction out of the 0-degree direction and the 90-degree direction or any other direction, and the profile area thereof can be obtained. Where the subject is not circular but is oval-shaped or can be approximated to an oval shape, X-ray projection data of the subject can be obtained free from lacking in the channel direction or deterioration in S/N ratio in some view directions if the aperture width of the X-ray beam in the channel direction is sufficient to some extent, and the profile area thereof can be obtained. When the subject is fully contained in the imaging field of view of the X-ray CT apparatus, the total profile area is constant in the case of a normal parallel beam. Also in the case of a fan beam, it can be considered approximately constant. For this reason, on the basis of the total profile area obtained by scout scanning, lacking parts of the projection data in the projection data obtained by the channel direction X-ray collimator can be supplemented by prediction, and a correct tomogram can be obtained for the region or area desired to be imaged. Also, even if the cause of the lack of some channels in projection data is a channel skip by or some trouble in the X-ray detector, correction can be made to carry out image reconstruction. Even if data on some channels in projection data are lacking or much noise is occurring on account of a substance which, present in the tomogram, hardly transmits X-rays (metal or the like), image reconstruction can be accomplished in higher picture quality if it is possible to make correction by replacement with smooth projection data maintaining the profile area.

In its seventh aspect, the invention provides an X-ray CT apparatus characterized in that, in any of the first through sixth aspects, it has X-ray data acquisition means in which the lack of some channels in projection data is attributable to the channel direction X-ray collimator; and image reconstructing means which carries out image reconstruction by figuring out the quantity of correction of X-ray projection data collected on the basis of positional information of the channel direction X-ray collimator and correcting the X-ray projection data accordingly.

The X-ray CT apparatus in the seventh aspect makes it possible, by having the channel-direction X-ray collimator, not to irradiate any non-region of interest with X-rays or, in other words, to realize a reduction in exposure to X-ray radiation by reducing unnecessary irradiation with X-rays in the channel direction. A reduction in exposure to X-ray radiation can be realized by so controlling the channel-direction X-ray collimator as to irradiate only region or area desired to be imaged with X-rays and enable irradiation with X-rays to be optimized.

Further in respect of image reconstruction, in the first through 13th aspects described above, even where some channels are lacking or the S/N ratio is poor, image reconstruction can be accomplished after making corrections by adding X-ray projection data at the time of image reconstruction.

In its eighth aspect, the invention provides an X-ray CT apparatus characterized in that, in any of the first through sixth aspects, it has X-ray data acquisition means in which the lack of some channels in projection data is attributable to the beam forming X-ray filter, and image reconstructing means which carries out image reconstruction by figuring out the quantity of correction of X-ray projection data collected on the basis of positional information of the beam forming X-ray filter and correcting the X-ray projection data accordingly.

In the X-ray CT apparatus in the eighth aspect, also the beam forming X-ray filter, like the channel-direction X-ray collimator, irradiates with X-rays the region of interest by only the X-ray aperture width centering on the X-ray beam position in a certain channel direction. Outside the X-ray aperture width, the dose of irradiation with X-rays is reduced, and the S/N ratio is deteriorated. For this reason, by using X-ray projection data of the subject obtained from scout images or the total profile area of the subject containing the X-ray profile of the whole subject and obtained from X-ray projection data of certain views, free from the lack of X-ray projection data or deterioration in S/N ratio, image reconstruction can be accomplished after making corrections by adding X-ray projection data at the time of image reconstruction even where some channels are lacking or the S/N ratio is poor.

In its ninth aspect, the invention provides an X-ray CT apparatus characterized in that, in any of the first through eight aspects, it has image reconstructing means which, using information on the profile area of scout images or the profile area of X-ray projection data of views not lacking in any channel, corrects and adds X-ray projection data of some channels lacking or deteriorated in S/N ratio so as to keep constant the profile area of the X-ray projection data of each view.

In the X-ray CT apparatus in the ninth aspect, when the subject is fully contained in the imaging field of view of the X-ray CT apparatus, the total profile area is constant in the case of a normal parallel beam. Also in the case of a fan beam, it can be considered approximately constant.

For this reason, by using the total profile area obtained by scout scanning or the total profile area of the subject containing the X-ray profile of the whole subject and obtained from X-ray projection data of certain views, free from the lack of X-ray projection data or deterioration in S/N ratio, correction can be made by adding X-ray projection data so that the profile area of X-ray projection data in each view direction is made equal to the total profile area and substantially constant in each view direction. In this way, image reconstruction can be accomplished after making corrections by adding X-ray projection data at the time of image reconstruction even where some channels are lacking or the S/N ratio is poor.

In its 10th aspect, the invention provides an X-ray CT apparatus characterized in that, in any of the first through ninth aspects, it has imaging condition setting means to set the region of interest desired to be imaged, and image reconstructing means which varies the position of X-ray projection data to be added and the profile area measure according to the position and scout images of the region of interest desired to be imaged or the positional relationship between the X-ray projection data of views not lacking in any channel and the profile area.

In the X-ray CT apparatus in the 10th aspect described above, in the 10th aspect, X-ray projection data can be corrected, when the profile area Sc of X-ray projection data in a certain view direction is smaller than the total profile area S, by adding X-ray projection data of S-Sc to both sides of the profile so as to make the profile area of X-ray projection data in each view direction equal to the total profile area and substantially constant in each view direction.

Especially where the region of interest desired to be imaged is set and that region of interest is not at the center of the whole imaging field of view, the range of parts of the profile which is deficient in X-ray projection data or deteriorated in S/N ratio varies on both sides dependent on the positions of views. For this reason, correction should be made while varying the area of the X-ray profile to be added from view to view.

This enables image reconstruction, even where some channels are lacking or the S/N ratio is poor, to be accomplished after making corrections by adding X-ray projection data at the time of image reconstruction.

In its 11th aspect, the invention provides an X-ray CT apparatus characterized in that, in the X-ray CT apparatus in the 10th aspect, it has X-ray data acquisition means having at least one of a channel direction X-ray collimator which tracks in the channel direction the region of interest desired to be imaged while acquisition X-ray projection data and a beam forming X-ray filter.

In the X-ray CT apparatus in the 11th aspect, the channel-direction X-ray collimator or the beam forming X-ray filter is subjected to positional control and aperture with control in the region of interest desired to be imaged so as to minimize irradiation with X-rays.

Further in this case, since either the outside of the region of interest is not irradiated with X-rays at all or reduced in the dose of irradiation with X-rays, exposure to radiation can be reduced.

In its 12th aspect, the invention provides an X-ray CT apparatus characterized in that, in the X-ray CT apparatus in the 11th aspect, it has X-ray data acquisition means which figures out in advance by calculation at least one of the channel position and the aperture width in the channel direction for each view or views at constant intervals for an region of interest of a preset region desired to be imaged of the subject, and subjects to feed forward control at least one of the channel direction X-ray collimator and the beam forming X-ray filter to match the channel position and the channel aperture width so figured out.

In the X-ray CT apparatus in the 12th aspect, since the channel position and the aperture width of the channel direction X-ray collimator or the beam forming X-ray filter in each view position are figured out in advance for a determined region of interest desired to be imaged, optimization of irradiation with X-rays can be achieved by aligning the channel-direction X-ray collimator or the beam forming X-ray filter therewith by feed forward control.

In its 13th aspect, the invention provides an X-ray CT apparatus characterized in that, in the X-ray CT apparatus in the 11th aspect, it has X-ray data acquisition means which looks at the output of the X-ray detector in each view or views at constant intervals, measures whether or not at least one of the channel direction X-ray collimator and the beam forming X-ray filter is in the correct position in the channel direction and has the correct aperture width in the channel direction, and subjects any deviations between the setpoints and the measurements to feedback control.

In the X-ray CT apparatus in the 13th aspect, it is possible to locate the position of the channel-direction X-ray collimator or the beam forming X-ray filter by reading the output of the X-ray detector and, if the channel direction X-ray collimator or the beam forming X-ray filter is off its set position, to subject any deviations between the setpoints and the measurements of the position in the channel direction to feedback control by a collimator controller, there making it possible to move the channel-direction X-ray collimator to a more correct position and achieve accurate control.

In its 14th aspect, the invention provides an X-ray CT apparatus characterized in that, in the X-ray CT apparatus in either the 12th or 13th aspect, it has image reconstructing means which, using the profile area of a scout or information on the profile area of X-ray projection data of a view not lacking any channel, corrects and adds X-ray projection data of some channels, outside the aperture width in the channel direction, lacking in some channel or deteriorated in S/N ratio, so as to make constant the profile area of the X-ray projection data of each view.

In the X-ray CT apparatus in the 14th aspect, the position control and aperture width control of the X-ray collimator or the beam forming X-ray filter is accomplished in accordance with the position and size of the region of interest desired to be imaged. It is possible to determine the position and range of the X-ray profile of the projection data of each view to be added by using information on the position and aperture with of the X-ray collimator or the beam forming X-ray filter then. By adding X-ray profiles in positions not irradiated with an X-ray beam and thereby making correction so that the profile area of the X-ray projection data of each view is made constant, an appropriate tomogram can be subjected to image reconstruction.

In its 15th aspect, the invention provides an X-ray CT fluoroscopic apparatus characterized in that it uses an X-ray CT imaging method in an X-ray CT apparatus according to any of the first through 14th aspects.

In the X-ray CT fluoroscopic apparatus in the 15th aspect, since the region of interest alone or the region of interest more concentratively is irradiated with X-rays by the channel-direction X-ray collimator or the beam forming X-ray filter, and other areas are not, or little, irradiated with X-rays, the exposure of the operator's hands to X-rays at the time of puncturing in X-ray CT fluoroscopy can be reduced.

In its 16th aspect, the invention provides an X-ray CT fluoroscopic apparatus wherein the channel direction X-ray collimator or the beam forming X-ray filter is fixed in the central part or near the central part in the channel direction, and low exposure to radiation is realized by making the central part of the image reconstruction area the region of interest and aligning the region of interest of the subject with the central part of the image reconstruction area.

In the X-ray CT fluoroscopic apparatus in the 16th aspect, in addition to the 15th aspect, the extents of positional control and aperture width control of the X-ray collimator or the beam forming X-ray filter are reduced by bringing the region of interest desired to be imaged to the central part of the whole imaging area, resulting in more stable control.

The X-ray CT apparatus and the X-ray CT fluoroscopic apparatus according to the invention give the effect of realizing an X-ray CT apparatus which can provide tomograms of higher picture quality by performing image reconstruction, even where projection data have become lacking in the channel direction, correcting projection data.

As another effect, they give the effect of realizing an X-ray CT apparatus which is equipped with at least either one of a channel-direction X-ray collimator and a beam forming X-ray filter which irradiates with X-rays only the region of interest of the region to be tomographed, tracks the region of interest of the region to be tomographed and performs tomography without irradiating the unnecessary area with X-rays or with reduced irradiation, and correcting on the basis of prediction from a scout image or characteristic parameters, of which one example is the profile area of projection data not lacking in X-ray projection data in the channel direction or not deteriorated in S/N ratio, X-ray projection data in any lacking part or deteriorated in S/N ratio to make possible imaging with reduced exposure to radiation.

As still another effect, they give the effect of realizing an X-ray CT fluoroscopic apparatus which limits the X-ray irradiated area with the channel-direction X-ray collimator or beam forming X-ray filter to reduce the exposure of the operator, especially the exposure of the operator's hands, to radiation at the time of puncturing in X-ray CT fluoroscopy.

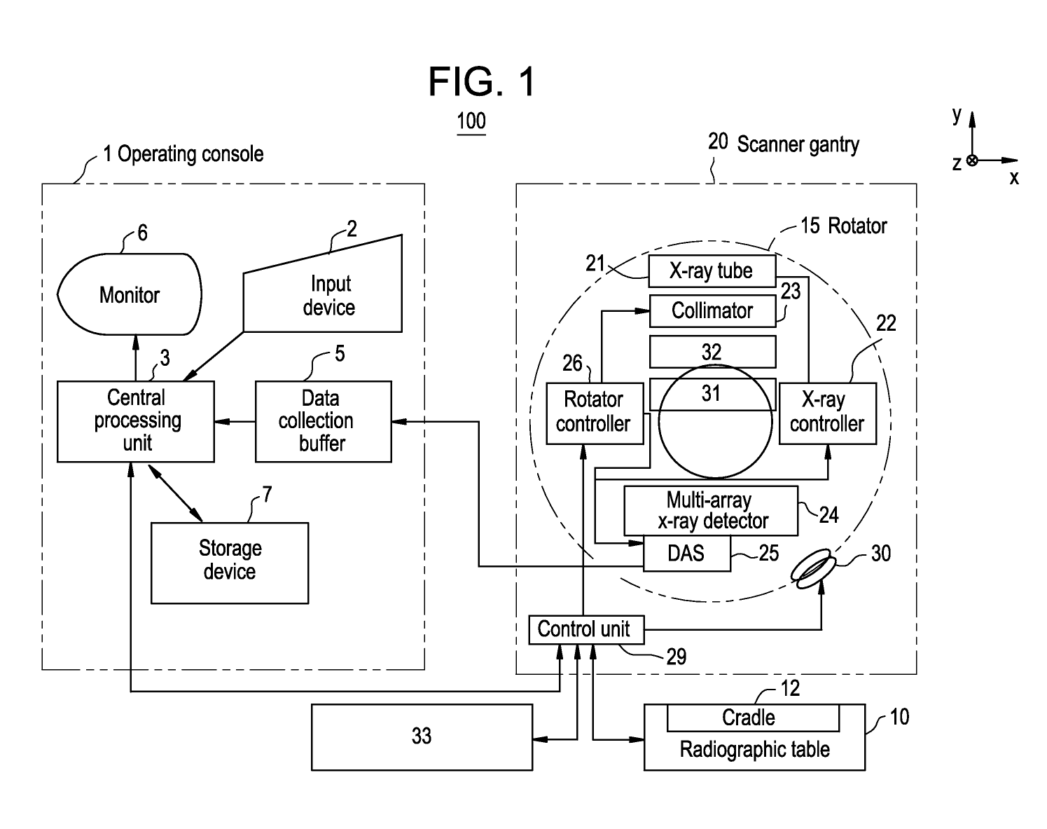

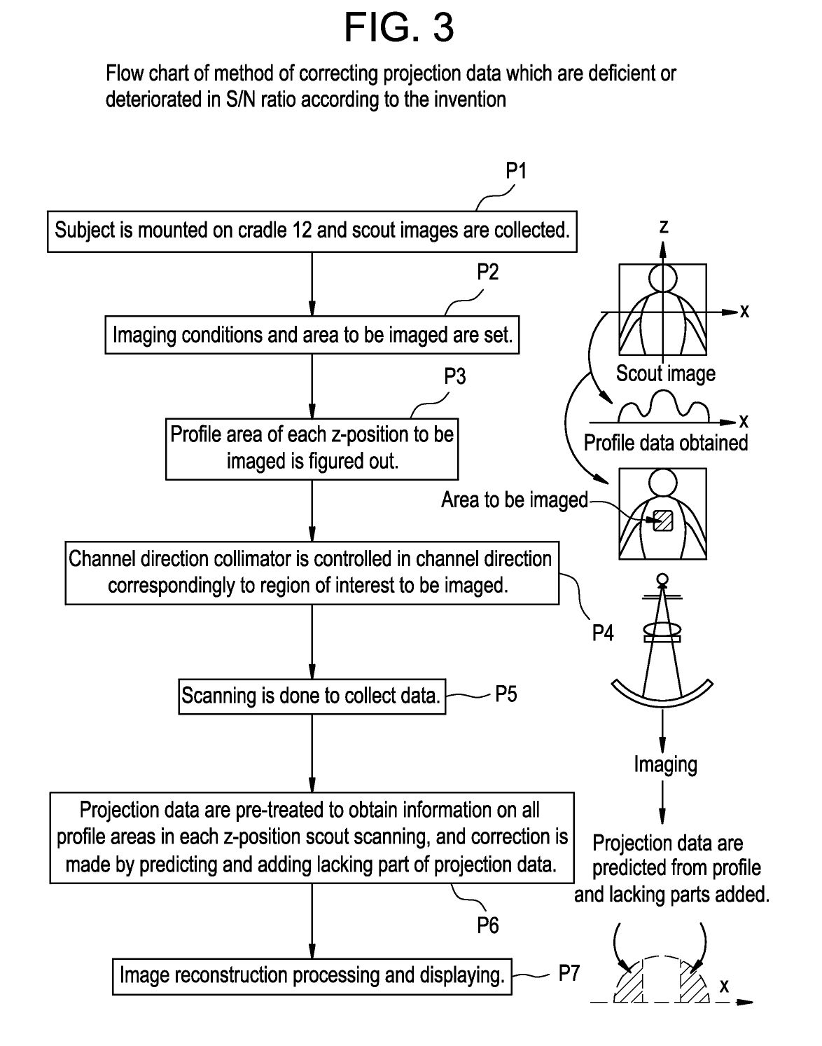

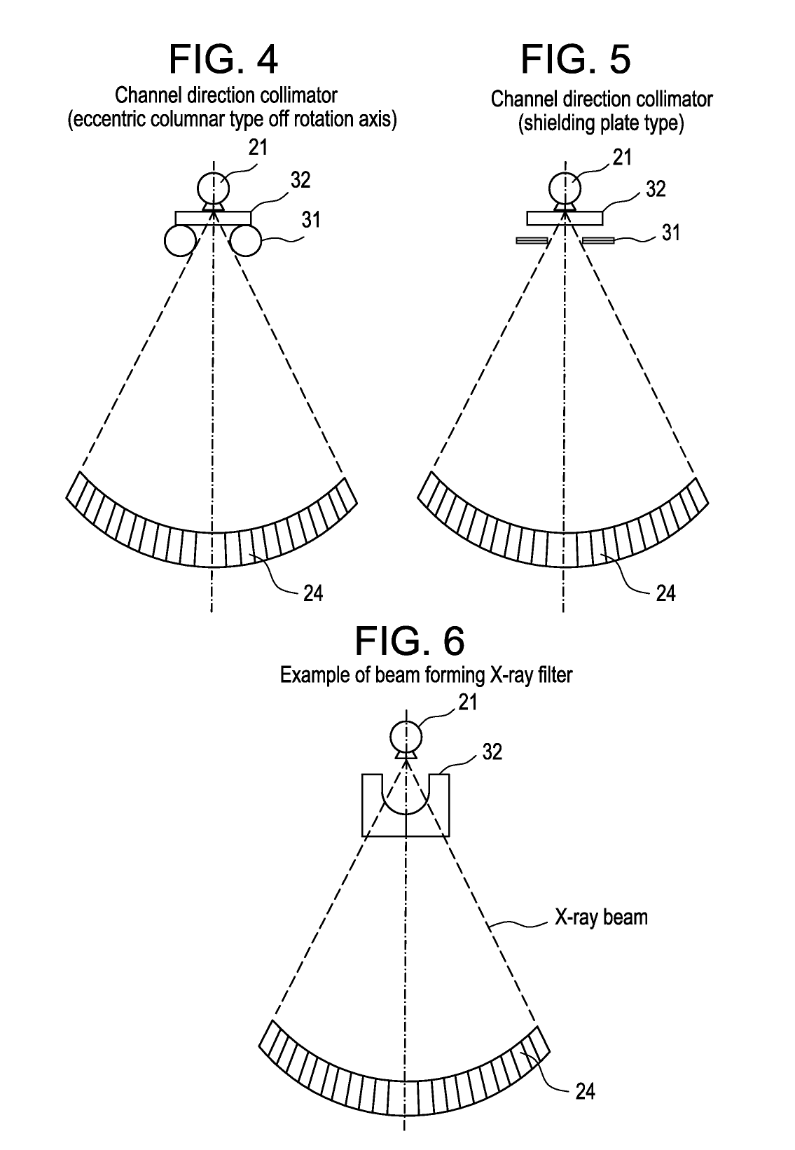

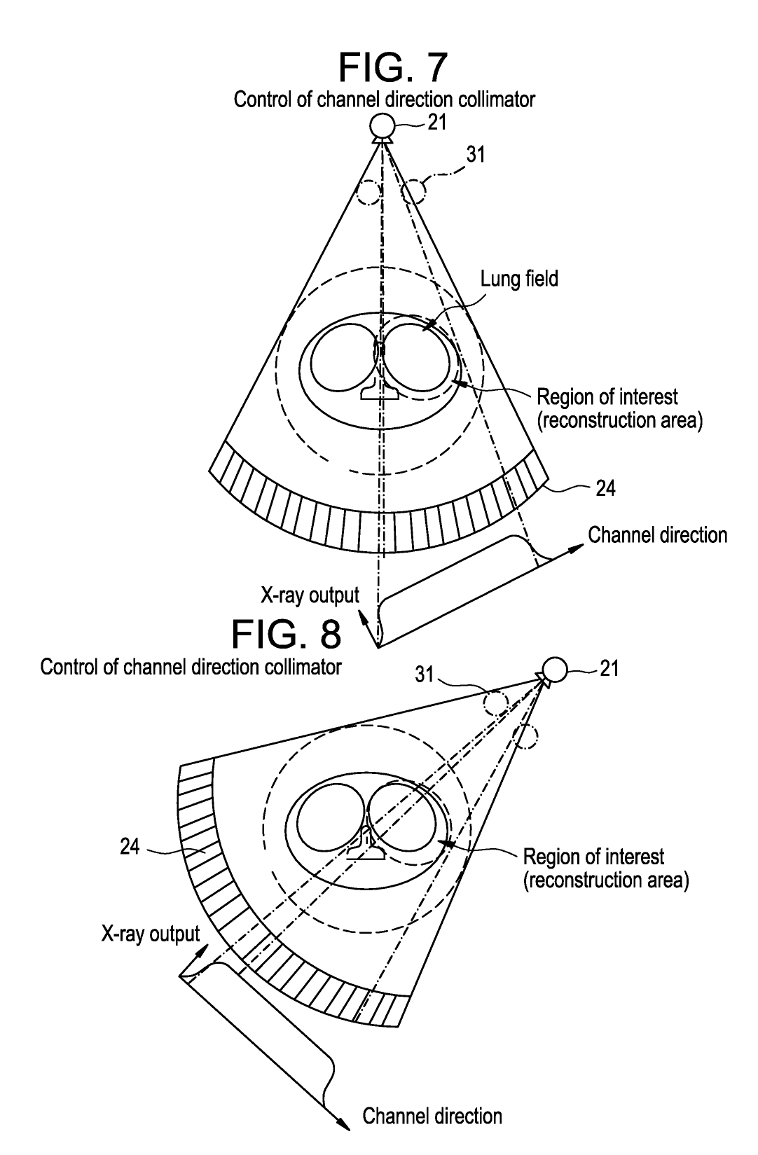

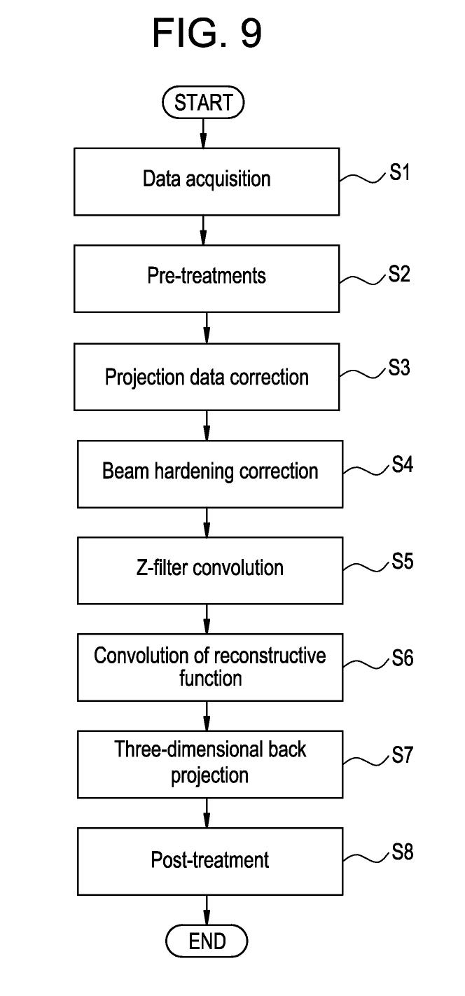

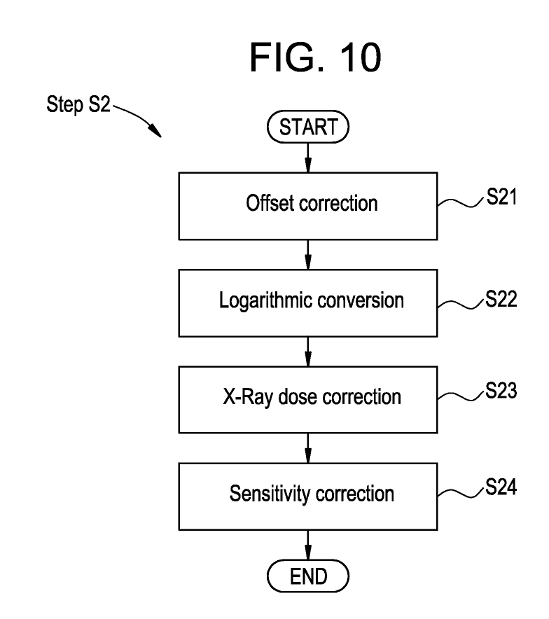

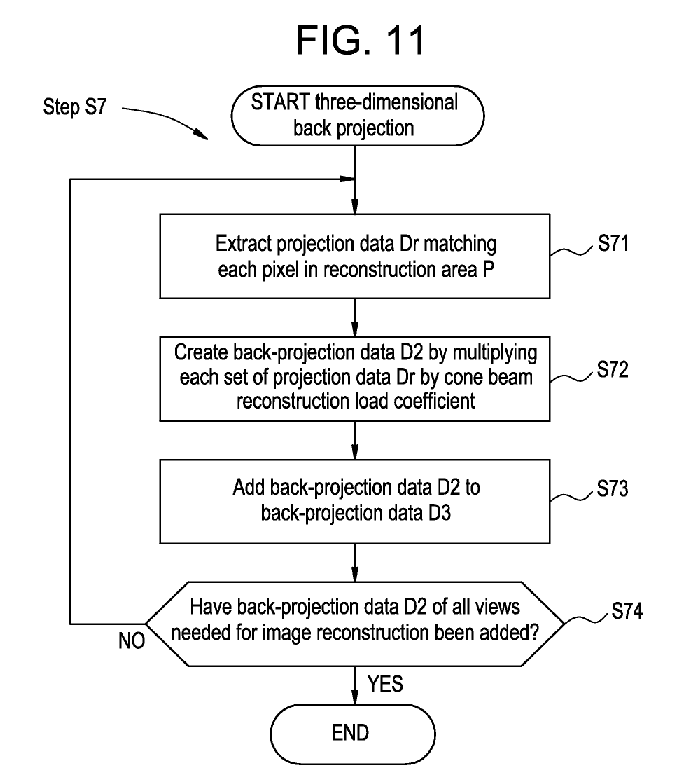

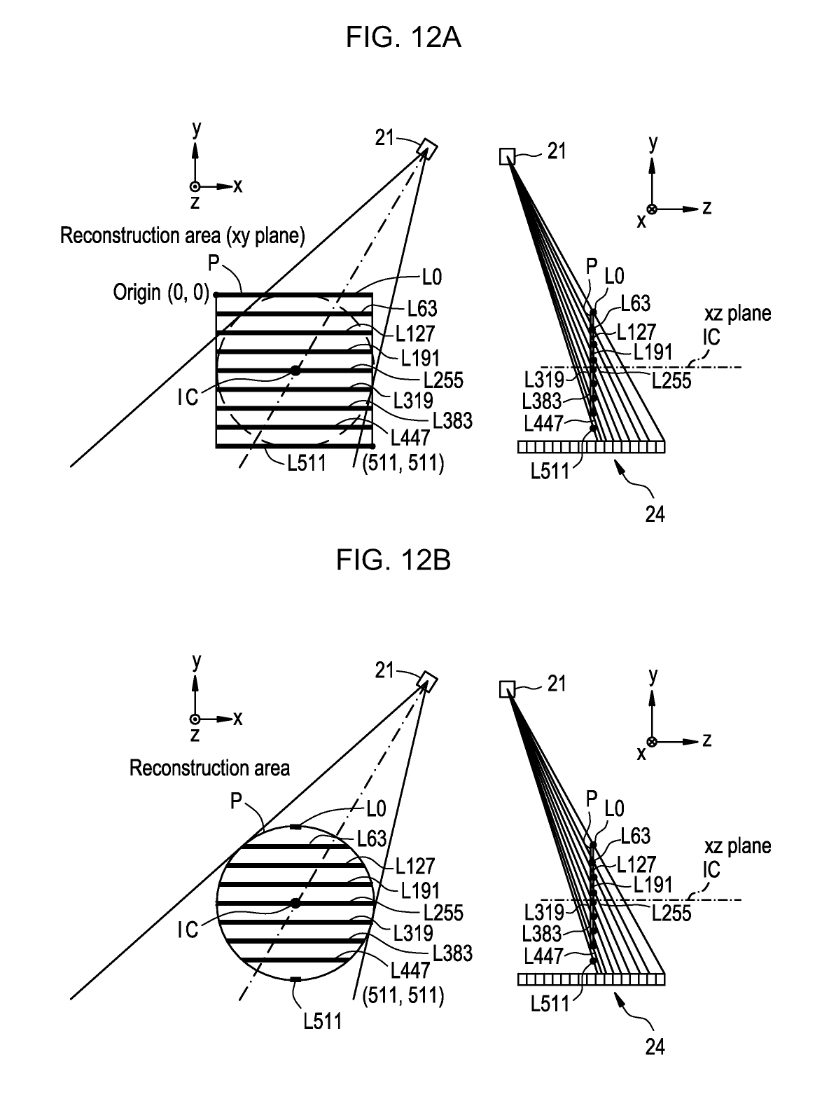

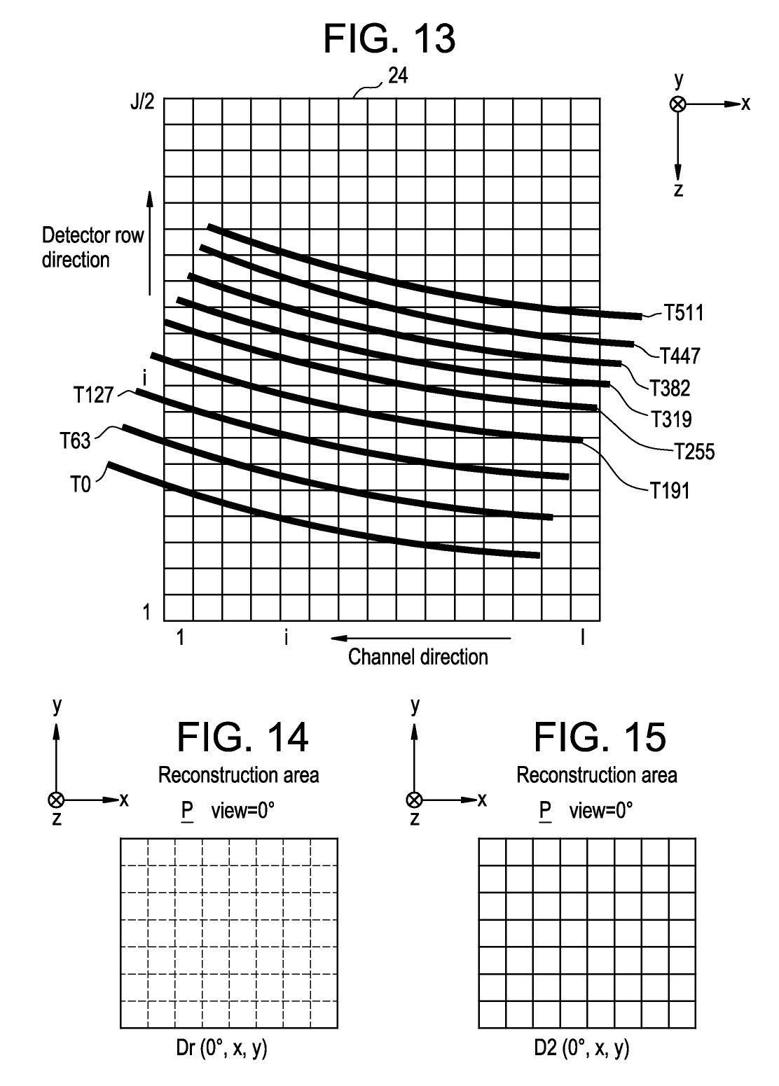

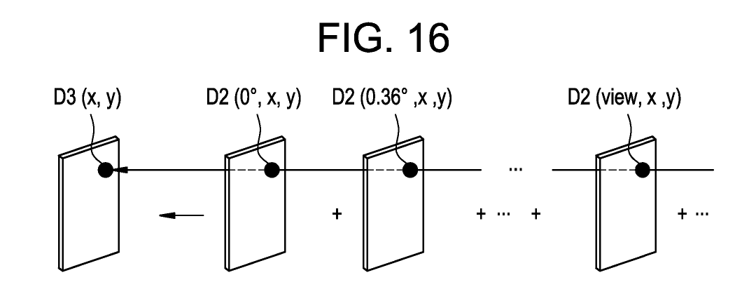

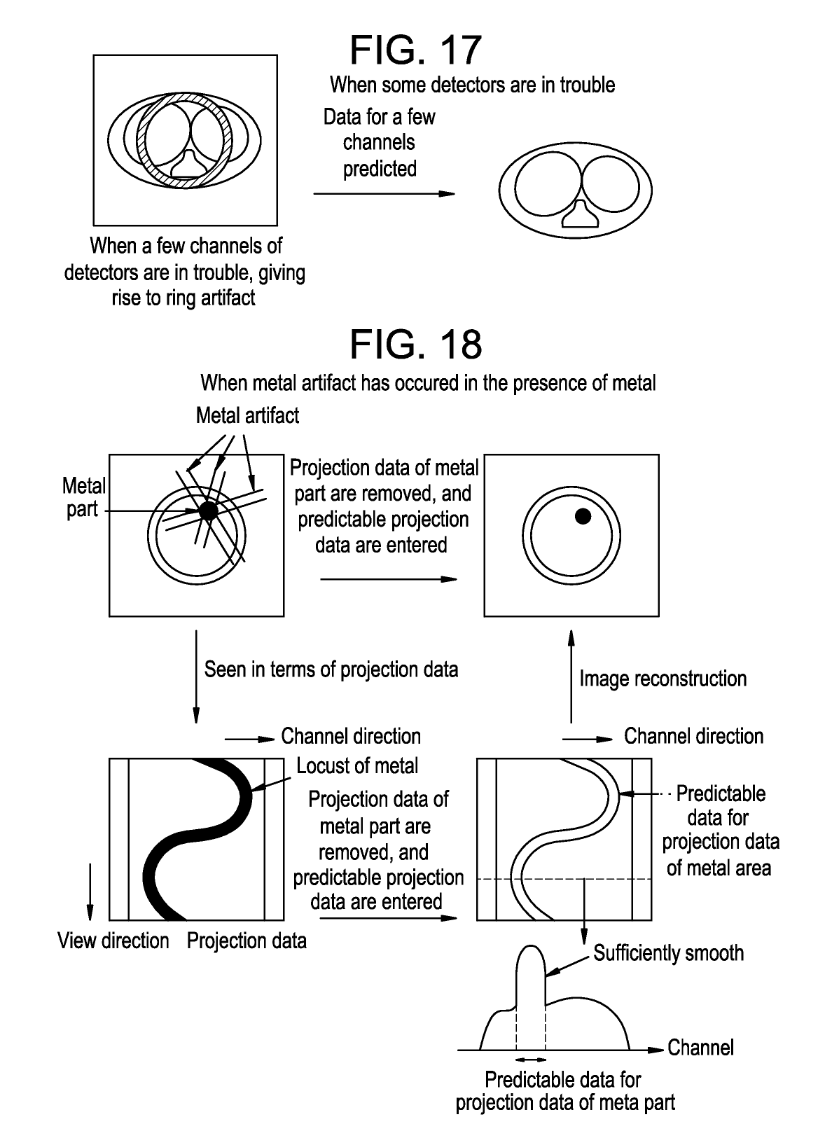

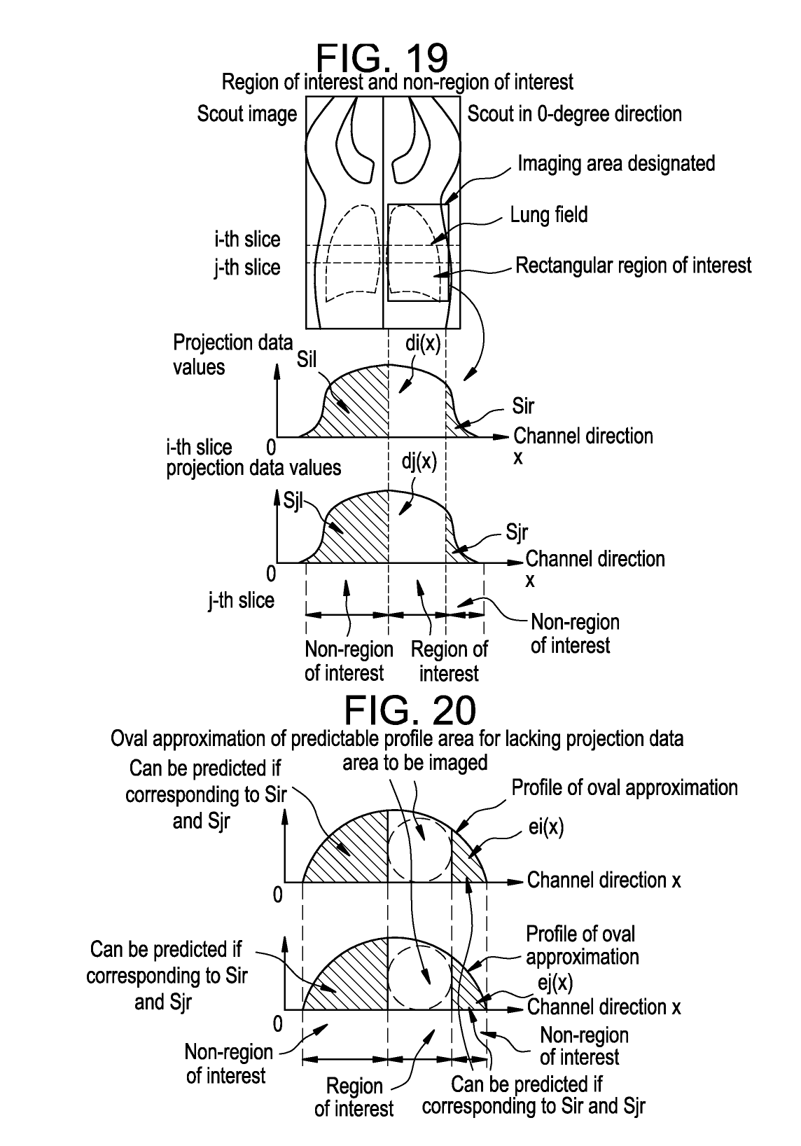

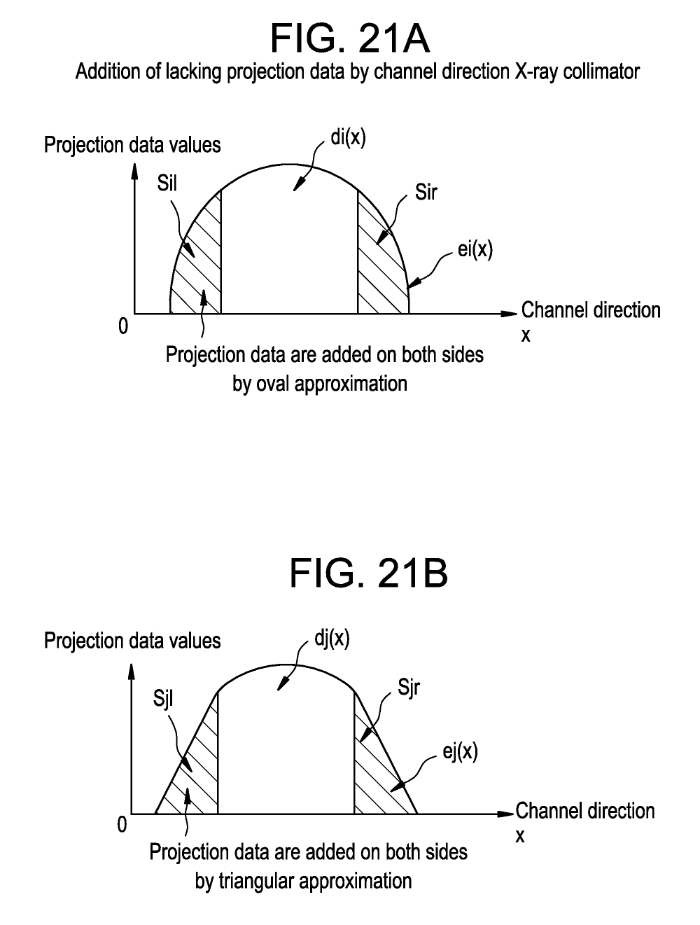

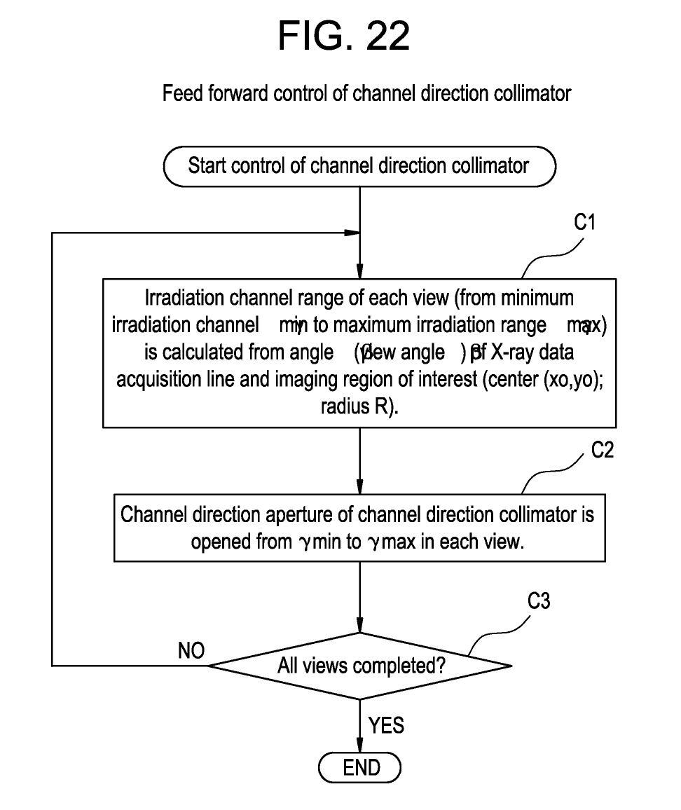

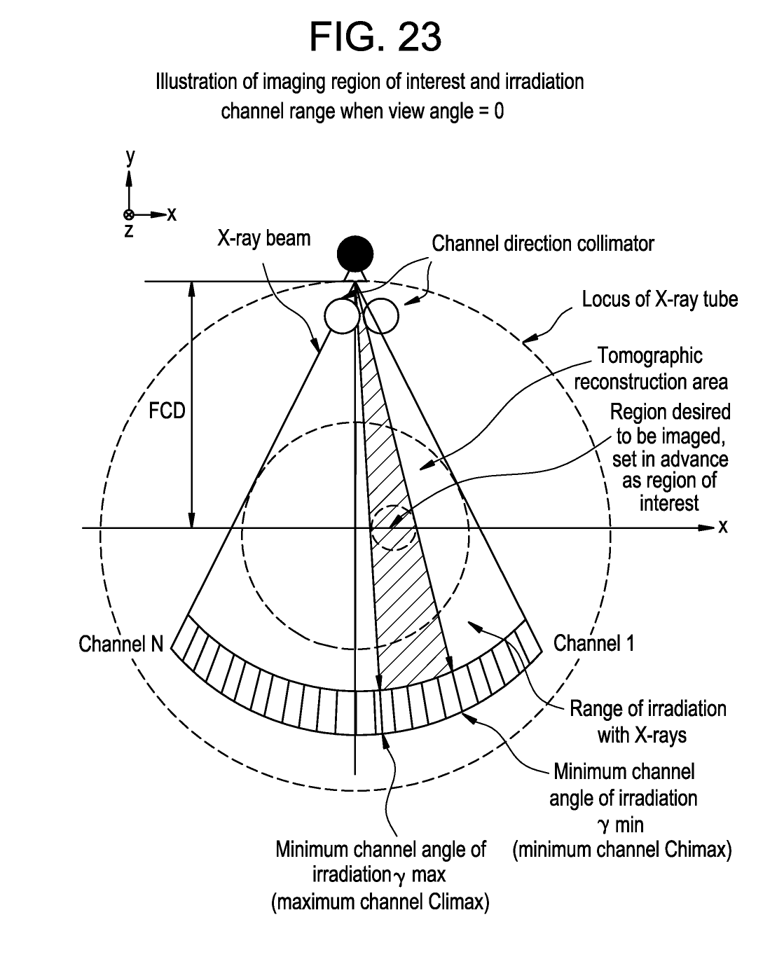

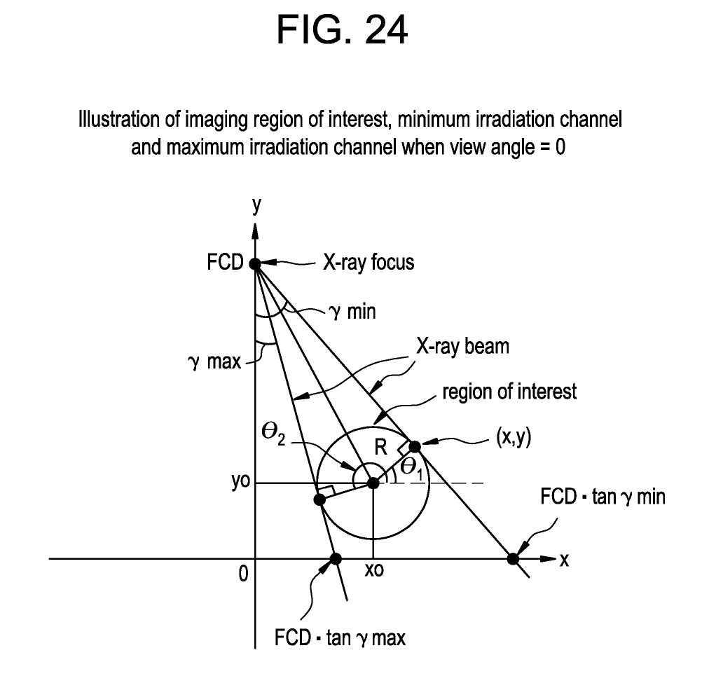

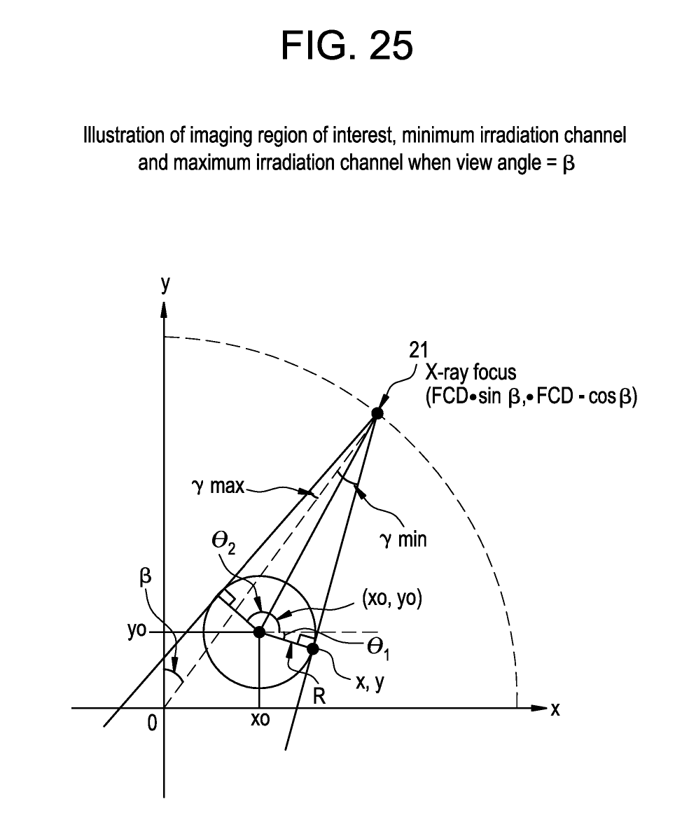

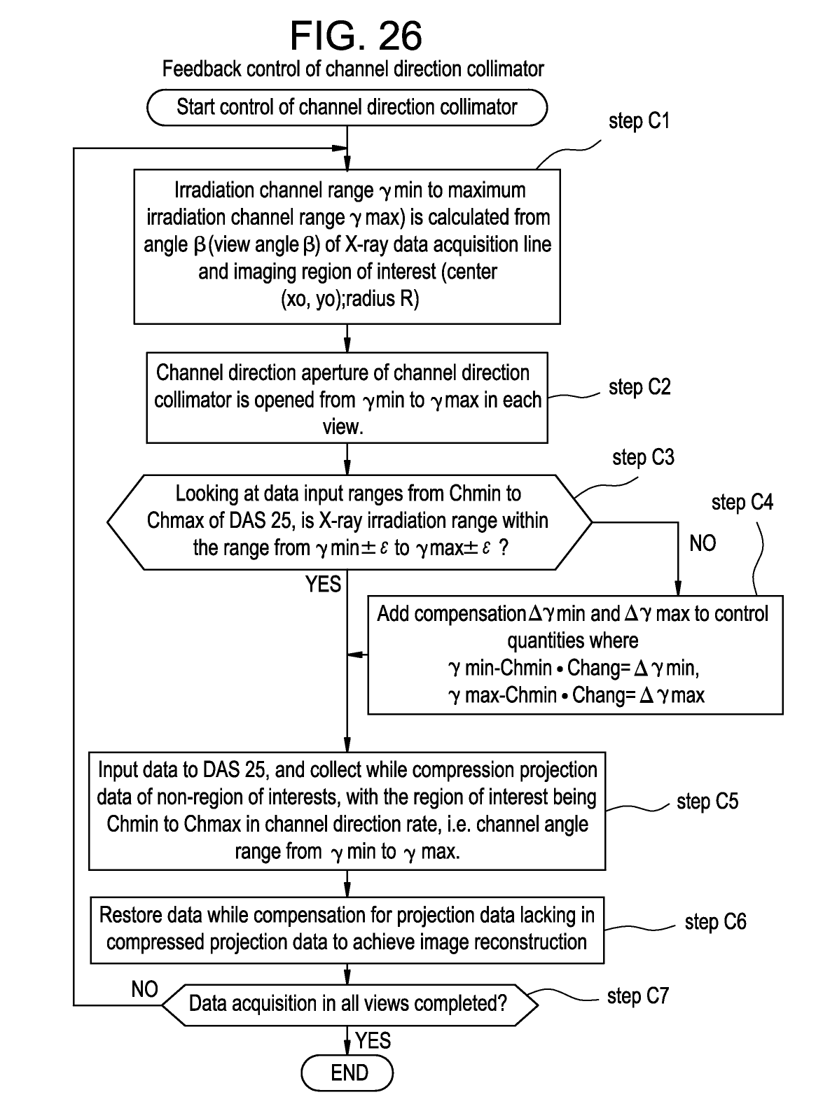

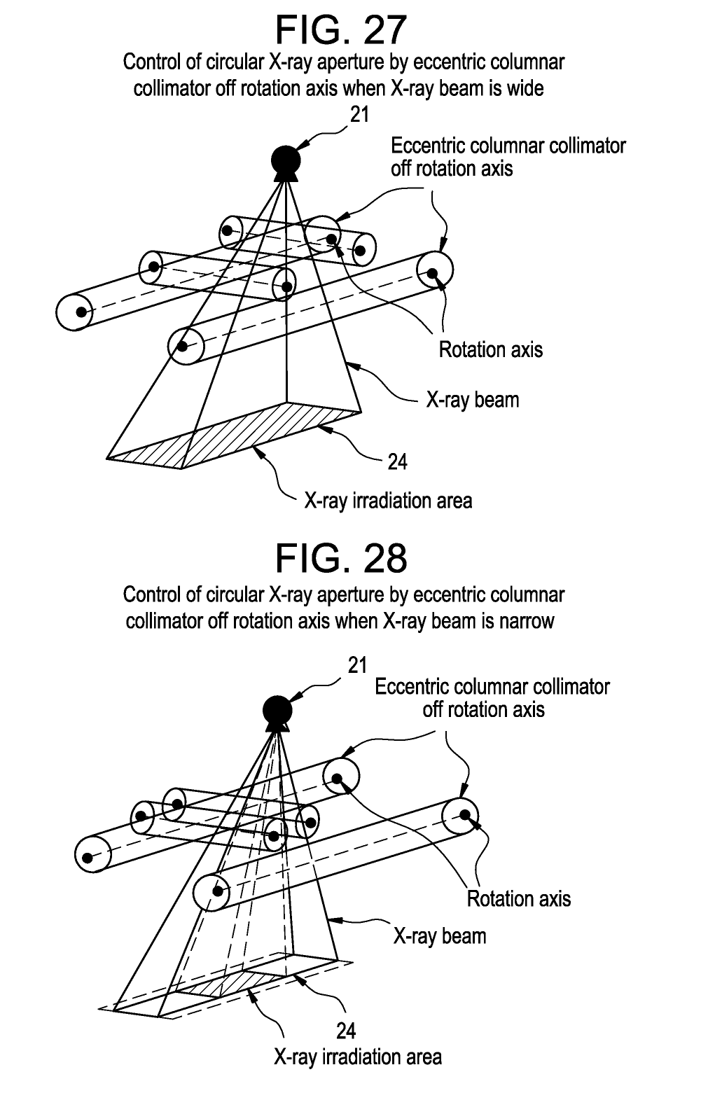

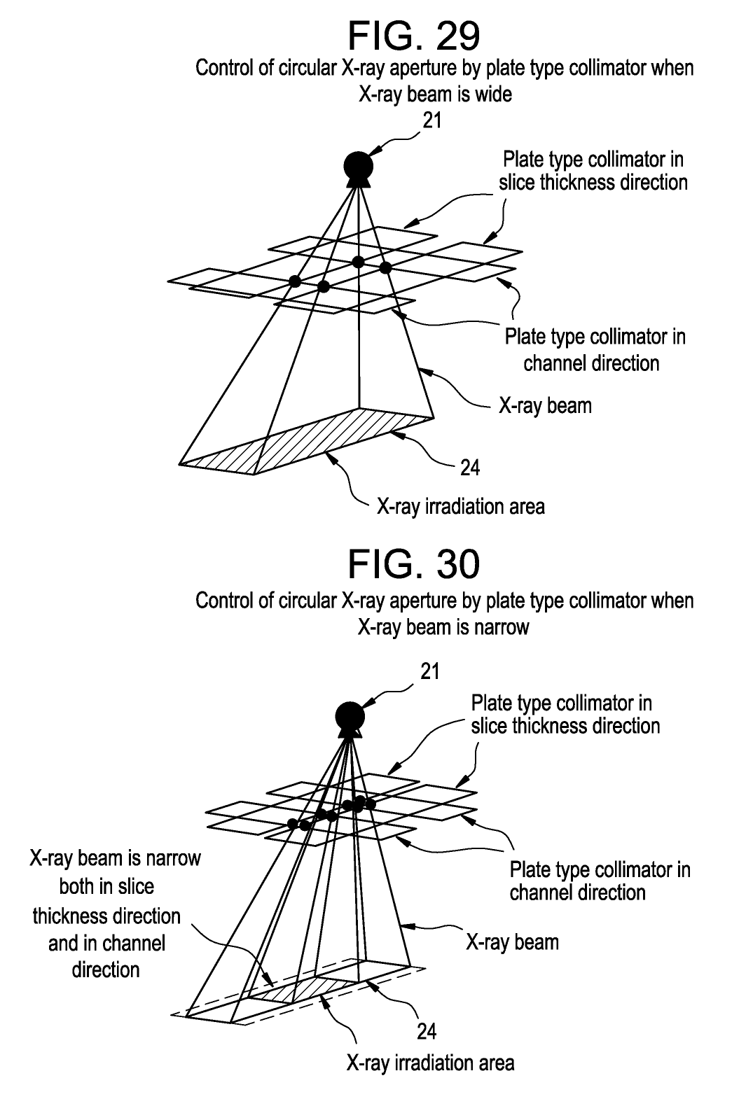

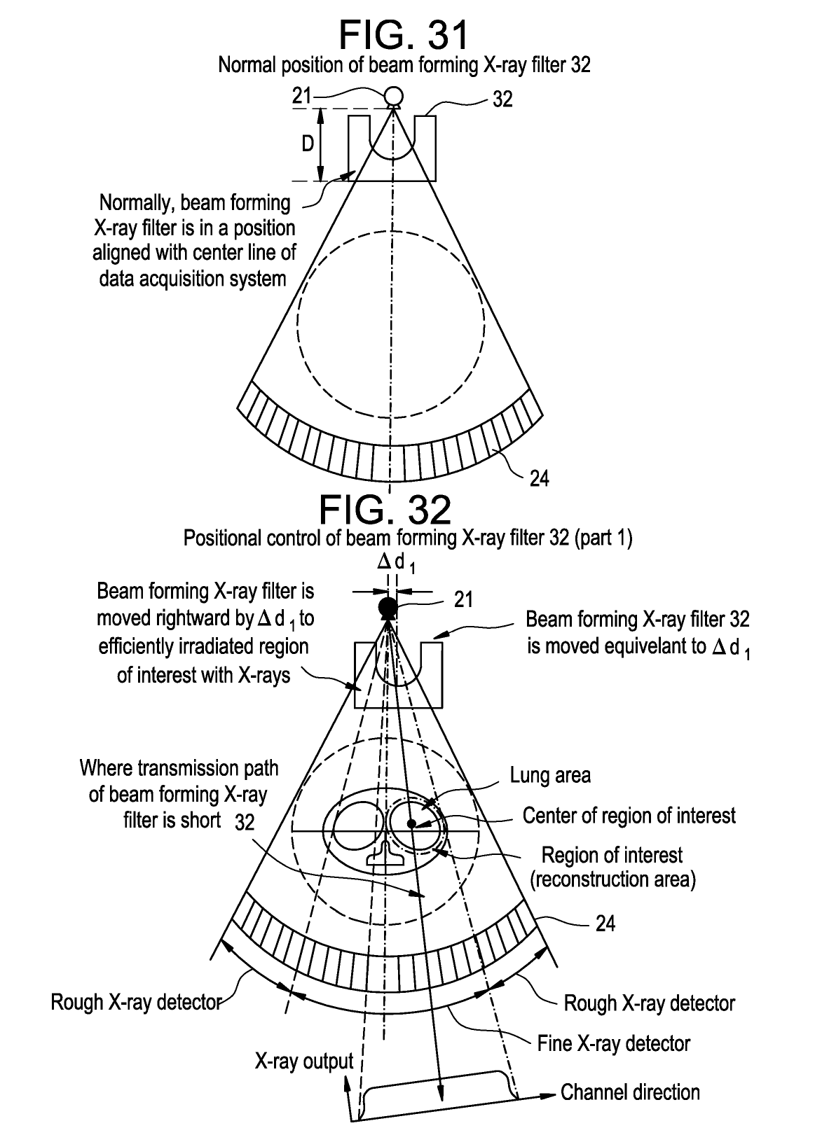

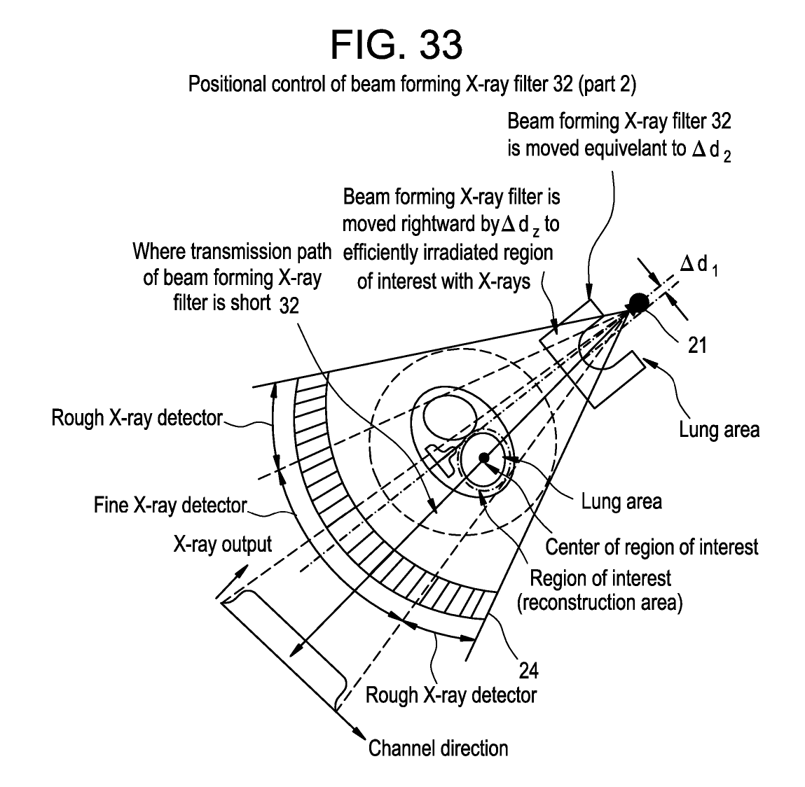

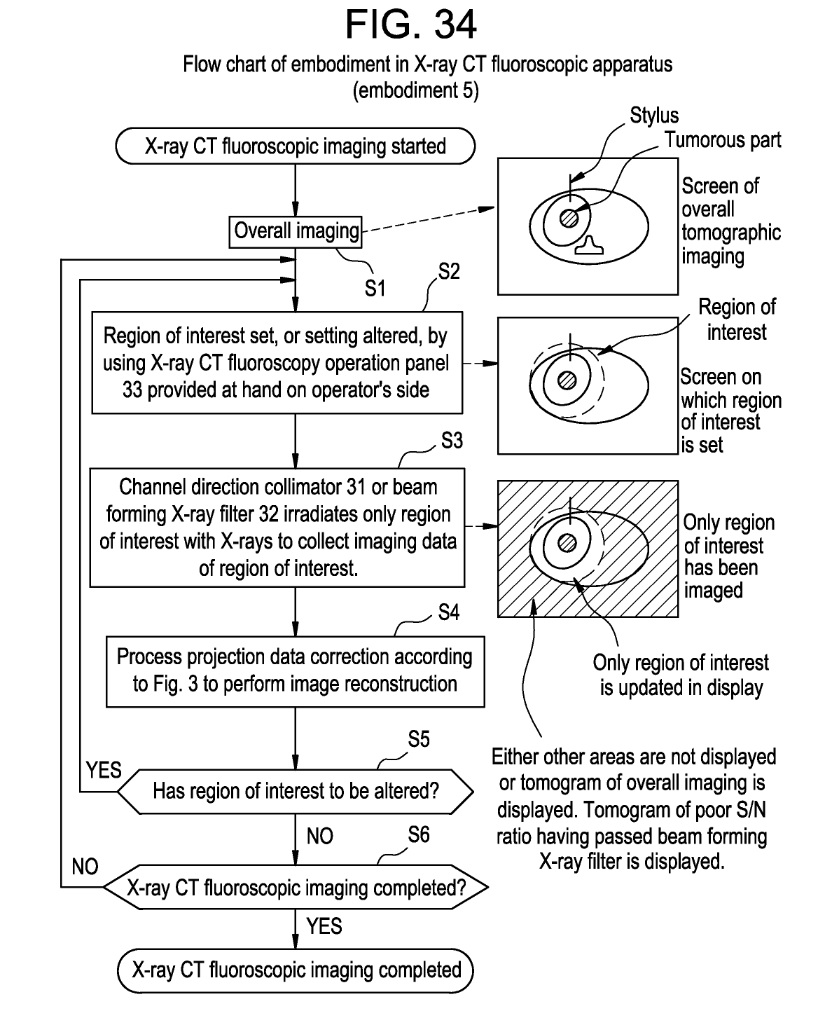

### BRIEF DESCRIPTION OF THE DRAWINGS

[FIG. 1] This is a block diagram of an X-ray CT apparatus in one mode for carrying out the present invention.

[FIG. 2] This is a diagram illustrating the rotation of an X-ray generating device (X-ray tube) and a multi-row X-ray detector.

[FIG. 3] This is a flow chart of a method of correcting projection data involving deficiency or deteriorated in S/N ratio.

[FIG. 4] This is a diagram showing a channel direction collimator (of an eccentric columnar type).

[FIG. 5] This is a diagram showing a channel direction collimator (of a shielding plate type).

[FIG. 6] This is a diagram showing an example of beam forming X-ray filter.

[FIG. 7] This is a diagram showing control by the channel direction collimator.

[FIG. 8] This is a diagram showing control by the channel direction collimator.

[FIG. 9] This is a diagram charting the flow of data acquisition and image reconstruction in one embodiment.

[FIG. 10] This is a flow charting showing details of pre-treatments.

[FIG. 11] This is a flow charting showing details of three-dimensional image reconstruction processing.

[FIGS. 12a and 12b] are conceptual diagrams showing the state of projecting a line on a reconstruction area in the X-ray transmitting direction.

[FIG. 13] This is a conceptual diagram showing a line projected on the plane of a detector.

[FIG. 14] This is a conceptual diagram showing the state of projecting projection data Dr (view, x, y) onto the reconstruction area.

[FIG. 15] This is a conceptual diagram showing back-projected pixel data D2 in the reconstruction area.

[FIG. 16] This is a diagram illustrating the state of obtaining back-projected data D3 by adding the back-projected pixel data D2 for all the views correspondingly to pixels.

[FIG. 17] This is a diagram illustrating a method of correcting projection data when part of a detector has trouble.

[FIG. 18] This is a diagram illustrating a method of correcting projection data when a metal artifact has occurred in the presence of metal.

[FIG. 19] This is a diagram showing a region of interest and non-region of interests.

[FIG. 20] This is a diagram showing prediction of lacking projection data.

[FIGS. 21a and 21b] are diagrams showing the addition of lacking projection data by a channel-direction X-ray collimator.

[FIG. 22] This is a diagram showing feed-forward control by the channel direction collimator.

[FIG. 23] This is a diagram illustrating the imaging region of interest and the irradiated range of channels when the view angle=0 degree.

[FIG. 24] This is a diagram illustrating the imaging region of interest, the minimum irradiated channels and the maximum irradiated channels when the view angle=0 degree.

[FIG. 25] This is a diagram illustrating the region of interest the minimum irradiated channels and the maximum irradiated channels when the view angle is β.

[FIG. 26] This is a diagram showing feedback control by the channel direction collimator.

[FIG. 27] This is a diagram showing control of a round X-ray aperture by a columnar X-ray collimator whose rotation axis is eccentric when the X-ray beam is wide.

[FIG. 28] This is a diagram showing control of the round X-ray aperture by the columnar X-ray collimator whose rotation axis is eccentric when the X-ray beam is narrow.

[FIG. 29] This is a diagram showing control of the round X-ray aperture by a planar X-ray collimator when the X-ray beam is wide.

[FIG. 30] This is a diagram showing control of the round X-ray aperture by the planar X-ray collimator when the X-ray beam is narrow.

[FIG. 31] This is a diagram showing the normal position of a beam forming X-ray filter 32.

[FIG. 32] This is a diagram showing positional control (part 1) of the beam forming X-ray filter 32.

[FIG. 33] This is a diagram showing positional control (part 2) of the beam forming X-ray filter 32.

[FIG. 34] This is a flow chart of an embodiment (Embodiment 5) in an X-ray CT fluoroscopic apparatus.

### DETAILED DESCRIPTION OF THE INVENTION

The present invention will be described in further detail below with reference to modes for carrying out as illustrated in drawings. Incidentally, this is nothing to limit the invention.

FIG. 1 is a configurative block diagram of an X-ray CT apparatus in one for carrying out the present invention. This X-ray CT apparatus 100 is equipped with an operation console 1, an imaging table 10 and a scanning gantry 20.

The operation console 1 is equipped with an input device 2 for accepting inputs by the operator, a central processing unit 3 for executing the processing of image reconstruction and the like, a data acquisition buffer 5 for collecting projection data acquired by the scanning gantry 20, a monitor 6 for displaying CT images reconstructed from the projection data, and a storage unit 7 for storing programs, data and X-ray CT images.

An input of imaging conditions is entered through this input device 2 and stored in the storage unit 7.

The imaging table 10 is equipped with a cradle 12 which places in and out a subject, whom it is mounted with, through the opening of the scanning gantry 20. The cradle 12 is lifted, lowered and moved along the table line by a motor built into the imaging table 10.

The scanning gantry 20 is equipped with an X-ray tube 21, an X-ray controller 22, a collimator 23 (collimator in the slice thickness direction), a multi-row X-ray detector 24, a DAS (Data Acquisition System) 25, a rotary unit 15, a rotary unit controller 26 for controlling the X-ray tube 21 and others rotating around the body axis of the subject, and a regulatory controller 29 for exchanging control signals and the like with the operation console 1 and the imaging table 10.

FIG. 2 illustrates the geometrical arrangement of the X-ray tube 21 and the multi-row X-ray detector 24. In the slice thickness direction X-rays are controlled by the collimator 23 (collimator in the slice thickness direction), and in the channel direction the X-rays are controlled by the channel direction collimator 31. Both in the slice thickness direction and in the channel direction, the X-ray aperture is controlled by rotating their axes of rotation center eccentric two exactly or approximately columnar objects made of a material which never or hardly transmits X-rays. The position and width of the X-ray aperture are controlled by moving independently in the slice direction and the channel direction two tabular X-ray shields made of a material which never or hardly transmits X-rays. An example of columnar X-ray shielding collimator eccentric in rotation axis is shown in FIG. 4, and an example of tabular X-ray shielding collimator is shown in FIG. 5. Further, how the aperture positions and the aperture widths of these collimators are shown in FIG. 27, FIG. 28, FIG. 29 and FIG. 30.

Further, in front of the X-ray tube 21 there is present beam forming X-ray filter 32. This beam forming X-ray filter 32 is an X-ray filter which is the thinnest in filter thickness at the center in the channel direction, does not absorb so much X-rays, and increases in filter thickness absorbing more and more X-rays toward peripheral channels. FIG. 6 shows an example thereof.

The X-ray tube 21 and the multi-row X-ray detector 24 rotate around the rotation center IC. The vertical direction being the y direction, the horizontal direction the x direction and the direction of the table movement perpendicular to them the z direction, the rotational plane of the X-ray tube 21 and the multi-row X-ray detector 24 is the xy plane. Further, the moving direction of the cradle 12 is the z direction.

FIG. 2 shows a view of the geometrical arrangement of the X-ray tube 21 and the multi-row X-ray detector 24 as seen from the xy plane.

The X-ray tube 21 generates an X-ray beam known as cone beam CB. When the direction of the center axis of the cone beam CB is parallel to the y direction, the view angle is supposed to be 0 degree.

The multi-row X-ray detector 24 has, for instance, 256 detector rows. Each detector row has, for instance, 1024 detector channels.

As shown in FIG. 2, after an X-ray beam leaving the X-ray focus of the X-ray tube 21 undergoes such spatial control by the beam forming X-ray filter 32 that more X-rays irradiate the center of the reconstruction area P and less X-rays irradiate the peripheries of the reconstruction area P, X-rays present within the reconstruction area P are absorbed by the subject, and transmitted X-rays are collected by the multi-row X-ray detector 24 as X-ray detector data.

As shown in FIG. 2, the X-ray beam leaving the X-ray focus of the X-ray tube 21 undergoes control by the X-ray collimator 23 in the slice thickness direction of the tomogram, namely in such a way that the X-ray beam width is D on the rotation center axis IC, and X-rays are absorbed by the subject present near the rotation center axis IC, and transmitted X-rays are collected by the multi-row X-ray detector 24 as X-ray detector data. Further, the channel direction collimator 31 controls the position and width of the X-ray beam in the channel direction.

Collected projection data following irradiation with X-rays are supplied from the multi-row X-ray detector 24 and subjected to A/D conversion by the DAS 25, and inputted to the data acquisition buffer 5 via a slip ring 30. The data inputted to the data acquisition buffer 5 are processed by the central processing unit 3 in accordance with a program in the storage unit 7 to be converted into a tomogram, which is displayed on the monitor 6.

FIG. 3 is a flow chart schematically showing the operation of the X-ray CT apparatus 100.

The following will be described regarding the present invention.

(1) When part of a detector has trouble (Embodiment 1)

(2) When metal is present (Embodiment 2)

(3) When an additional channel direction collimator is disposed and channel direction collimators are controlled according to the magnitude of the FOV desired to be reconstructed (Embodiment 3)

Whereas the shielding cylinder type (eccentric columnar collimator type off the rotation axis) (FIG. 4) or of the shielding plate type (the tabular collimator type (FIG. 5) are conceivable for the collimator in Embodiment 3, either is applicable according to the invention. While collimator control in the z-direction (the slice thickness direction) was accomplished having the DAS 25 read z-channel data, collimator control in the channel direction is carried out by finding out in advance, the position of X-rays to be brought into incidence on the multi-row X-ray detector 25, which is determined by the angle β (the view angle β) of the X-ray data acquisition line and the position and magnitude of the region of interest to be imaged, and subjecting the aperture position and the aperture width of the channel direction collimator to feed-forward control on that basis. Also, feedback control in the channel direction is performed as required with the value of the main detector channel of the DAS 25 which collects projection data (see FIG. 7 and FIG. 8).

The progress of the performance of DAS-controlling CPUs and collimator-controlling CPUs seems to have made it sufficiently free from problems to carry out calculations for feedback control of the channel direction collimator aperture on the basis of the data read from the main detector channel of the multi-row X-ray detector 24. If the patient is too obese to ensure a high enough S/N ratio of X-ray data, only feedback control may be performed according to the position of the channel direction collimator predictable from the position and magnitude of the imaged field of view.

Drive systems, such as a pulse motor, for controlling the operation of the collimator in this case are considered fast enough in response.

In the overall flow charted in FIG. 3, the following operation will take place in any of Embodiments 1, 2 and 3.

At step P1, the subject is mounted on the cradle 12 and aligned. The subject mounted on the cradle 12 undergoes alignment of the reference point of each region to the central position of the slice light of the scanning gantry 20. After that, data of scout images are collected. Scout images are usually taken at 0 degree and 90 degrees, but only a 90-degree scout image is taken for some regions, including the head for instance. Details of scout imaging will be described afterwards.

At step P2, after setting the imaging conditions, the area to be imaged is set on the scout image. Regarding the imaging conditions, usually imaging is carried out while displaying on the scout image the position and size of the tomogram to be picked up. In this case, X-ray dose information on a full round of helical scanning, variable-pitch helical scanning, conventional scanning (axial scanning) or cine-scanning is displayed. Further in cine-scanning, if the number of revolutions or time length is inputted, X-ray dose information for the number of revolutions or the time length inputted in that region of interest will be displayed.

At step P3, the profile area of each z-position to be imaged is figured out.

At step P4, the channel direction collimator is controlled in the channel direction correspondingly to the region of interest to be imaged.

At step P5, scanning is done to collect data.

At step P6, projection data are pre-treated to obtain information on all the profile areas in each z-position scout scanning, and correction is made by predicting and adding with the channel direction collimator the projection data part lacking in the peripheries in the channel direction.

At step P7, image reconstruction is processed and an image is displayed by using the projection data corrected by supplementing the lacking part.

FIG. 9 is a flow chart outlining the data acquisition and processing for tomography and scout imaging by the X-ray CT apparatus 100.

At step S1, first, helical scanning is performed while rotating the X-ray tube 21 and the multi-row X-ray detector 24 around the object of imaging and linearly moving the cradle 12 on the table, and projection data are collected by adding the z-direction position z table (view) to projection data D0 (view, j, i) represented by the linear movement position z of the table, the view angle view, the detector row number j and the channel number i. In variable-pitch helical scanning, not only data acquisition in helical scanning is performed at a constant speed but also data acquisition is carried out during acceleration and during deceleration.

Further, in conventional scanning (axial scanning) or cine-scanning, X-ray detector data are collected by rotating the data acquisition line one round or a plurality of round while keeping the cradle 12 on the imaging table 10 fixed in a certain z-direction position. X-ray detector data are further collected by rotating the data acquisition line one round or a plurality of round as required after moving to the next z-direction position.

On the other hand, in scout imaging, X-ray detector data are collected while keeping the X-ray tube 21 and the multi-row X-ray detector 24 fixed and linearly moving the imaging table 10.

At step S2, projection data D0 (view, j, i) are pre-treated to be converted into projection data. The pre-treatments comprise offset correction at step S21, logarithmic conversion at step S22, X-ray dose correction at step S23 and sensitivity correction at step S24 as shown in FIG. 10.

In scout imaging, by displaying the pre-treated X-ray detector data matched with the pixel size in the channel direction and the pixel size in the z-direction, which is the linear moving direction of the cradle matched with the display pixel size of the monitor 6, the scout image is completed.

Step S3 is processing to correct projection data which are deficient or deteriorated in S/N ratio.

Step S3 will be described below with respect to Embodiments 1, 2 and 3 with reference to FIG. 17, FIG. 18 and, FIG. 19 through FIG. 21.

### Embodiment 1

As shown in FIG. 17, when part of a detector has trouble, if the number of channels in trouble is small, it will have little impact on the profile area, and therefore the following simple correction will be sufficient.

Projection data being represented by d(i, j, k) (where i is the channel, j is the view and k is the row), if

$$[ Mathematical Expression 1 ]$$

$$Th 1 > {\sum }_{1} d ( i , j , k )$$

holds with respect to a certain threshold Th1, that i channel will be deemed to be in trouble.

Where the channel in trouble is any of i₁ to iₙ interpolation is performed with data of i₁−1 channel and iₙ+1 channel. It is provided that m=0 to n−1 holds.

$$[ Mathematical Expression 2 ]$$

$$d ( {i}_{1} + m , j , k ) = d ( {i}_{1} - 1 , j , k ) + ( \begin{matrix}d ( {i}_{n} + 1 , j , k ) - \\ d ( {i}_{1} - 1 , j , k )\end{matrix} ) \times \frac{m + 1}{n + 1}$$

### Embodiment 2

Where a metal artifact has occurred in the presence of metal as shown in FIG. 18, projection data on the metal are removed and predictable projection data are entered. As the values of predictable projection data in this case, large enough values as projection data on metal which are sufficiently smooth projection data and would not overflow in the subsequent image reconstructing calculations would be acceptable.

### Embodiment 3

As shown in FIG. 19 through FIG. 21, when X-rays from other regions than what is to be imaged with the channel-direction X-ray collimator, projection data in the shielded parts need to be predicted.

Feed-forward control by the channel direction X-ray collimator will be described with reference to the flow chart of FIG. 22.

At step C1, as shown in FIG. 23, the angle range on the multi-row X-ray detector 24 to be irradiated with X-rays (from the minimum irradiation channel γmin to the maximum irradiation channel γmax) or the channel range is figured out by calculation according to the angle β (the view angle β) of the X-ray data acquisition line, comprising the X-ray tube 21, the multi-row X-ray detector 24 and the DAS 25, and the size and position of the imaging region of interest (e.g. a circular region of interest of a radius R around the center (x0, y0)).

Here,
the position of the X-ray tube bulb
x=FCD·sin θ
y=FCD·cos θ [Mathematical Expression 3]

where θ is the view angle and FCD (Focus Center Distance of x-rays).

At step C2, the channel direction collimator (which may either be an eccentric columnar collimator or a shielding plate type collimator) opens from the minimum irradiation channel γmin to the maximum irradiation channel γmax.

At step C3, it is checked whether collimator control in the channel direction and data acquisition for all the scanned views of the planned imaging has been completed.

Incidentally, the relationship among the minimum irradiation channel γmin and the maximum irradiation channel γmax, the X-ray data acquisition line, comprising the X-ray tube 21, the multi-row X-ray detector 24 and the DAS25 and the channel direction collimator in the foregoing is shown in FIG. 23.

Further, the relationship among the imaging region of interest when the view angle is 0, the minimum irradiation channel and the maximum irradiation channel is as described below and shown in FIG. 24.

For instance, where the position of the circular-shaped imaging region of interest is (x0, y0), the radius is R and the view angle is 0 degree, namely the X-ray focus is at (0, FCD), the following will hold (where FCD is Focus Center Distance of X-rays).

Thus,

$$[ Mathematical Expression 4 ]$$

$$\{ \begin{matrix}y = - \frac{1}{tan γ} \cdot x + FCD & ( Formula 1 ) \\ x = xo + R \cdot cos θ & ( Formula 2 ) \\ y = yo + R \cdot sin θ & ( Formula 3 )\end{matrix}$$

From Formulas 1, 2 and 3:

$$tan γ = \frac{- x}{FCD - y}$$

$$\begin{matrix}γ = {tan}^{- 1} ( \frac{- x}{FCD - y} ) \\ = {tan}^{- 1} ( \frac{- xo - R \cdot sin θ}{FCD - yo - R \cdot cos θ} )\end{matrix}$$

The maximum value of (then is (max and the minimum value of (is (min.

$$\begin{matrix}γ max = {tan}^{- 1} ( \frac{xo + R \cdot sin {θ}_{2}}{FCD - yo - R \cdot cos {θ}_{2}} ) & ( Formula 4 ) \\ γ min = {tan}^{- 1} ( \frac{xo + R \cdot sin {θ}_{1}}{FCD - yo - R \cdot cos {θ}_{1}} ) \\ Hence , \\ [ Mathematical Expression 5 ] \\ γmax = {tan}^{- 1} ( \frac{xo + R \cdot sin {θ}_{2}}{FCD - yo - R \cdot cos {θ}_{2}} ) \\ γmin = {tan}^{- 1} ( \frac{xo + R \cdot sin {θ}_{1}}{FCD - yo - R \cdot cos {θ}_{1}} ) & ( Formula 5 )\end{matrix}$$

Further, the relationship among the imaging region of interest when the view angle is β, the minimum irradiation channel, and the maximum irradiation channel is as described below as shown in FIG. 25.

For instance, where the position of the circular-shaped imaging region of interest is (x0, y0), the radius is R and the view angle is 0 degree, namely the X-ray focus is at (FCD·sin β,FCD·cos β), the following will hold (where FCD is Focus Center Distance of X-rays).

Thus,

$$Mathematical Expression 6 ]$$

$$\{ \begin{matrix}y = - \frac{1}{tan ( y - β )} ( x - FCD \cdot sin β ) + FCD \cdot cos β & ( Formula 11 ) \\ x = xo + R \cdot sin θ & ( Formula 12 ) \\ y = yo + R \cdot cos θ & ( Formula 13 )\end{matrix}$$

From Formulas 4, 5 and 6:

$$tan ( γ - β ) = - \frac{FCD \cdot sin β - x}{FCD \cdot cos β - y}$$

$$γ = β - {tan}^{- 1} ( \frac{FCD \cdot sin β - xo - R \cdot sin θ}{FCD \cdot cos β - yo - R \cdot cos θ} )$$

The maximum value of γ then is γmax and the minimum value of γ is γmin.

$$\begin{matrix}γmax = β - {tan}^{- 1} ( \frac{FCD \cdot sin β - xo - R \cdot sin θ1}{FCD \cdot cos β - yo - R \cdot cos θ1} ) & ( Formula 14 ) \\ γmin = β - {tan}^{- 1} ( \frac{FCD \cdot sin β - xo - R \cdot sin θ2}{FCD \cdot cos β - yo - R \cdot cos θ2} ) \\ Hence , \\ [ Mathematical Expression 7 ] \\ γmax = β - {tan}^{- 1} ( \frac{FCD \cdot sin β - xo - R \cdot sin θ1}{FCD \cdot cos β - yo - R \cdot cos θ1} ) \\ γmin = β - {tan}^{- 1} ( \frac{FCD \cdot sin β - xo - R \cdot sin θ2}{FCD \cdot cos β - yo - R \cdot cos θ2} ) & ( Formula 15 )\end{matrix}$$

Next, feedback control by the channel-direction X-ray collimator is shown in FIG. 26.

At step C1, as at step C1 in FIG. 22, the angle range on the multi-row X-ray detector 24 to be irradiated with X-rays (from the minimum irradiation channel γmin to the maximum irradiation channel γmax) or the channel range is figured out by calculation according to the angle β (the view angle () of the X-ray data acquisition line, comprising the X-ray tube 21, the multi-row X-ray detector 24 and the DAS25, and the size and position of the imaging region of interest (e.g. a circular region of interest of a radius R around the center (x0, y0)).

At step C2, as at step C2 in FIG. 22, the channel direction collimator (which may either be an eccentric columnar collimator or a shielding plate type collimator) opens from the minimum irradiation channel (min to the maximum irradiation channel (max.

At step C3, the range of data irradiated with X-rays is figured out by looking at data in the DAS 25. If the input range of data irradiated with is from Chmin to Chmax, it is checked if this corresponds to the minimum irradiation channel (min to the range from the maximum irradiation channel (max figured out at step C1.

If the error is within a minute range of ±ε, it will be considered acceptable, but if this error range is exceeded, the process will go to step C4.

At step C4, correction quantities Δγmin and Δγmax are added to the controlled variables, where γmin−Chmin·Chang=Δγmin, and γmax−Chmax·Chang=Δγmax. This is followed by advancing to step C5.

At step C5, data are inputted to the DAS 25 and, with the region of interest spanning the channel direction range Chmin to Chmax, namely the channel angle range Tmin to Tmax, data are collected while compressing projection data in the non-region of interest.

At step C6, image reconstruction is carried out by restoring the compressed projection data while supplementing the lacking projection data.

At step C7, it is checked whether or not data acquisition has been completed for all the views and, if it has not been, the process returns to step C1, and collimator control in the channel direction and data acquisition are continued.

In this case, oval approximation is carried out according to the profile area and the width profile in the channel direction. As shown in FIG. 20 and FIG. 21. On the basis of the positional relationship between the oval-approximated profile and the area desired to be imaged, projection data Sil and Sir added to the left and right sides of the area desired to be imaged are known from the intercepted X-ray data on the i-th slice in each direction. By adding these Sil and Sir to the left and right of the projection data to carry out image reconstruction, a tomogram of higher picture quality can be obtained.

At step S4, projection data D1 (view, j, i) having undergone correction after the pre-treatment are subjected to beam hardening correction. The beam hardening correction at S4 can be expressed in, for instance, a polynomial form as represented below, with the projection data having undergone sensitivity correction at S24 of the pre-treatment S2 being represented by D1 (view, j, i) and the data after the beam hardening correction at S4 by D11 (view, j, i).
D11(view,j,i)=D1(view,j,i)·(Bo(j,i)+B₁(j,i)·D1(view,j,i)+B₂(j,i)·D1(view,j,i)²) [Mathematical Expression 8]

Since each j rows of detectors can be subjected to beam hardening correction independently of others then, if the tube voltage of each data acquisition line differs from others depending on imaging conditions, differences in detector characteristics from row to row can be compensated for.

At step S5, the projection data D11 (view, j, i) having undergone beam hardening correction are subjected to filter convolution, by which filtering is done in the z-direction (the row direction).

Thus, the data D11 (view, j, i) (i=1 to CH, j=1 to ROW) of the multi-row X-ray detector having undergone beam hardening correction after the pretreatment at each view angle and on each data acquisition line are subjected to, for instance, filtering whose row-direction filter size is five rows.
(w₁(i),w₂(i),w₃(i),w₄(i),w₅(i)), [Mathematical Expression 9]

provided that

$${\sum }_{k - 1}^{5} {w}_{k} ( i ) = 1$$

The corrected detector data D12(view, j, i) will be as follows.

[Mathematical Expression 10]

$$D 12 ( view , j , i = {\sum }_{k - 1}^{5} ( D 11 ( view , j + k - 3 , i ) \cdot {w}_{k} ( j ) )$$

Incidentally, the maximum channel width being supposed to be CH and the maximum row value being ROW, the following will hold.
D11(view,−1,i)=D11(view,0,i)=D11(view,1,i)
D11(view,ROW,i)=D11(view,ROW+1,i)=D11(view,ROW+2,i) [Mathematical Expression 11]

On the other hand, the slice thickness can be controlled according to the distance from the center of image reconstruction by varying the row-direction filter coefficient from channel to channel. Since the slice thickness is usually greater in the peripheries than at the center of reconstruction in a tomogram, the slice thickness can be made substantially uniform whether in the peripheries or at the center of image reconstruction by so differentiating the row-direction filter coefficient between the central part and the peripheries that the range of the row-direction filter coefficient is varied more greatly in the vicinities of the central channel and varied more narrowly in the vicinities of the peripheral channel.

By controlling the row-direction filter coefficient between the central channels and the peripheral channels of the multi-row X-ray detector 24 in this way, the control of the slice thickness can also be differentiated between the central part and the peripheries. By slightly increasing the slice thickness with the row-direction filter, substantial improvements can be achieved in terms of both artifact and noise. The extent of improvement of artifact and that of noise can be thereby controlled. In other words, a tomogram having undergone three-dimensional image reconstruction, namely picture quality in the xy plane, can be controlled. Another possible embodiment, a tomogram of a thin slice thickness can be realized by using deconvolution filtering for the row-direction (z-direction) filter coefficient.

Further, X-ray projection data of the fan beam are converted into X-ray projection data of the parallel beam as required.

At step S6, convolution of the reconstructive function is performed. Thus, the result of Fourier transform is multiplied by the reconstructive function to achieve inverse Fourier transform. In the convolution of reconstructive function at S6, data after the convolution of z-filter being represented by D12, data after the convolution of reconstructive function by D13 and the reconstructive function to be convoluted by Kernel (j), the processing to convolute the reconstructive function can be expressed in the following way.
D13(view,j,i)=D12(view,j,i)*Kernel(j) [Mathematical Expression 12]

Thus, since the reconstructive function Kernel (j) permits independent convolution of the reconstructive function on each j rows of detectors, differences in noise characteristics and resolution characteristics from one row to another can be compensated for.

At step S7, the projection data D13 (view, j, i) having undergone convolution of the reconstructive function are subjected to three-dimensional back-projection to obtain back-projected data D3 (x, y). The image to be reconstructed is reconstructed into a three-dimensional image on a plane perpendicular to the z-axis and the xy plane. The following reconstruction area P is supposed to be parallel to the xy plane. This three-dimensional back-projection will be described afterwards.

At step S8, the back-projected data D3 (x, y, z) are subjected to post-treatments including image filter convolution and CT value conversion to obtain a tomogram D31 (x, y).

In the image filter convolution as post-treatment, with the data having gone through three-dimensional back-projection being represented by D31 (x, y, z), the data having gone through image filter convolution by D32 (x, y, z) and the image filter by Filter (z):
D32(x,y,z)=D31(x,y,z)*Filter(z) [Mathematical Expression 13]

Thus, since the reconstructive independent convolution of the reconstructive function is possible on each j rows of detectors, differences in noise characteristics and resolution characteristics from one row to another can be compensated for.

The tomogram that is obtained is displayed on the monitor 6.

FIG. 11 is a flow chart showing details of the three-dimensional back-projection process (step S7 in FIG. 9).

In this embodiment, the image to be reconstructed is reconstructed into a three-dimensional image on a plane perpendicular to the z-axis and the xy plane. The following reconstruction area P is supposed to be parallel to the xy plane.

At step S71, note is taken on one view out of all the views needed for image reconstruction of a tomogram (namely 360-degree views or “180-degree+fan angle” views), and projection data Dr corresponding to the pixels in the reconstruction area P are extracted.

As shown in FIGS. 12(a) and (b), a square area of 512×512 pixels parallel to the xy plane being supposed to be the reconstruction area P, and a pixel row L0 of y=0, a pixel row L63 of y=63, a pixel row L 127 of y=127, a pixel row L191 of y=191, a pixel row L255 of y=255, a pixel row L319 of y=319, a pixel row L383 of y=383, a pixel row L447 of y=447 and a pixel row L511 of y=511, all parallel to the x-axis, being taken as rows, if projection data on lines T0 through T511 are extracted as shown in FIG. 13, wherein these pixel rows L0 through L511 are projected on the plane of the multi-row X-ray detector 24 in the X-ray transmitting direction, they will constitute projection data Dr (view. x, y) of pixel rows L0 through L511. It is provided, however, that x and y matches pixels (x, y) in the tomogram.

Whereas the X-ray transmitting direction is determined by the geometrical positions of the X-ray focus of the X-ray tube 21, the pixels and the multi-row X-ray detector 24, since the z-coordinate z (view) of the projection data D0 (view, j, i) are known as the z-direction of the linear table movement Z table (view) attached to the projection data, the X-ray transmitting direction can be accurately figured out in the data acquisition geometric system of the X-ray focus and the multi-row X-ray detector even if the projection data D0 (view, j, i) are obtained during acceleration or deceleration.

Incidentally, if part of the lines goes out of the plane of the multi-row X-ray detector 24 as does, for instance, the line TO resulting from the projection of the pixel row L0 onto the plane in the multi-row X-ray detector 24 in the X-ray transmitting direction, the matching projection data Dr are set to “0”. If they go out in the z-direction, it will be figured out by extrapolating projection data Dr (view, x, y).

In this way, projection data Dr (view, x, y) matching the pixels of the reconstruction area P can extracted as shown in FIG. 14.

Referring back to FIG. 11, at step S72, projection data Dr (view,

x. y) are multiplied by a cone beam reconstruction weighting coefficient to create projection data D2 (view, x, y) shown in FIG. 15.

The cone beam reconstruction weighting coefficient w (i, j) here is as follows. In reconstructing a fan beam image, the following relationship holds where y is the angle which a straight line linking the focus of the X-ray tube 21 and a pixel g (x, y) forms with respect to the center axis of the X-ray beam where view=βa and the view opposite thereto is view=βb.
βb=βa+180°−2γ [Mathematical Expression 14]

With the angles formed by the X-ray beam passing the pixel g (x, y) on the reconstruction area P and the X-ray beam opposite thereto with respect to the reconstruction plane P being represented by βa and βb, the back-projected data D2 (0, x, y) are figured out by adding after multiplication with reconstruction weighting coefficients βa and βb. In this case, the following holds.
D2(0,x,y)=ωa·D2(0,x,y)_{—}a+ωb·D2(0,x,y)_{—}b [Mathematical Expression 15]

where D2 (0, x, y)_a are supposed to be the back-projected data of view βa and D2 (0, x, y)_b, the back-projected data of view βb.

Incidentally, the sum of the mutually opposite beams of cone beam reconstruction weighting coefficients is:
ωa+ωb=1 [Mathematical Expression 16]

By adding the products of multiplication by cone beam reconstruction weighting coefficients, the cone angle artifact can be reduced.

For instance, reconstruction weighting coefficients ωa and ωb obtained by the following formulas can be used. In these formulas, ga is the weighting coefficient of the view βa and gb, the weighting coefficient of the view βb.

Where ½ of the fan beam angle is γmax, the following holds.
ga=f(γmax,αa,βa)
gb=′f(γmax,ab,βb)
xa=2·ga^{q}/(ga^{q}+gb^{q})
xb=2·gb^{q}/(ga^{q}+gb^{q})
wa=xa²·(3−2xa)
wb=xb²·(3−2xb) [Mathematical Expression 17]

(For instance, q=1 is supposed.)

For instance, if max[ ] is supposed to be a function taking up what is greater in value as an example of ga and gb, the following will hold.
ga=max[0,{(π/2+γmax)−|βa|}]·|tan(αa)|
gb=max[0,{(π/2+γmax)−|βb|}]·|tan(αb)| [Mathematical Expression 18]

In the case of fan beam image reconstruction, each pixel of the reconstruction area P is further multiplied by a distance coefficient. The distance coefficient is (r1/r0)² where r0 is the distance from the focus of the X-ray tube 21 the detector row j and the channel i of the multi-row X-ray detector 24 matching the projection data Dr and r1, the distance from the focus of the X-ray tube 21 to a pixel matching the projection data Dr on the reconstruction area P.

In the case of parallel beam image reconstruction, it is sufficient to multiply each pixel only by the cone beam reconstruction weighting coefficient w (i, j).

At step S73, projection data D2 (view, x, y) are added, correspondingly to pixels, to back-projected data D3 (x, y) cleared in advance as shown in FIG. 16.

At step S74, steps 61 through S63 are repeated for all the views repeated for CT image reconstruction (namely 360-degree views or “180-degree+fan angle” views) to obtain back-projected data D3(x, y) as shown in FIG. 16.

Incidentally, the reconstruction area P may as well be a circular area as shown in FIGS. 12 (c) and (d).

### Embodiment 4

Whereas Embodiment 3 was described with reference to the channel direction X-ray collimator 31, the use of the beam forming X-ray filter 32 as shown in FIG. 31 could give a similar effect.

FIG. 31 shows the normal position of the beam forming X-ray filter, namely when the quantity of movement in the channel direction is 0.

FIG. 32 and FIG. 33 show cases in the quantity of movement of the beam forming X-ray filter is Δd₁, and Δd₂, respectively. In this case, the control can be so accomplished that the straight line linking the center of the region of interest and the focus of X-rays overlaps the X-ray transmission path of the beam forming X-ray filter 32 constituting the shortest straight line.

To achieve their overlapping:
γₘₑₐₙ=(γₘₐₓ+γₘᵢₙ)/2 [Mathematical Expression 19]

With the distance from the X-ray focus to the beam forming filter being represented by D as shown in FIG. 31, the following holds.
Δdi=D·tan(γₘₑₐₙ)

(where Δdi=Δd₁ or Δd₂)

### Embodiment 5

A case in which the present invention is used in an X-ray CT fluoroscopic apparatus is shown in FIG. 34. First at step S1, a whole tomogram is imaged.

Next at step S2, the region of interest desired to be imaged is set on the tomogram imaged at step S1. When setting this region of interest, the operator present in a scan room in which the scanning gantry 20 is installed sets the region of interest by using an X-ray CT fluoroscopy operation panel 33 provided at hand.

Next at step S3, the channel direction collimator 31 or a shape X-ray collimator 32 irradiates with X-rays while tracking the region of interest or its center in the channel direction to collect projection data in the region of interest.

Next at step S4, correction of projection data based on the whole profile area as shown in FIG. 3 is carried out, and the corrected projection data are subjected to image reconstruction.

Next at step S5, it is checked whether or not the region of interest needs to be altered.

Next at step S6, it is checked whether or not X-ray fluoroscopic imaging has been completed.

The X-ray CT apparatus 100 described above, by the X-ray CT apparatus or X-ray CT imaging method according to the invention, has an effect to reduce the exposure of the subject to radiation with its channel direction X-ray collimator compared with the conventional multi-row X-ray detector, X-ray CT apparatus or flat panel X-ray CT apparatus.

Incidentally, the image reconstruction method may be the usual three-dimensional image reconstruction method according to the already known Feldkamp method. It may even be some other three-dimensional image reconstructing method. It need not be three-dimensional image reconstruction, but conventional two-dimensional image reconstruction could provide a similar effect.

Further, though row-direction (z-direction) filters differing in coefficient from row to row are convoluted in this embodiment, filters not in the row-direction (z-direction) could also provide a similar effect.

Also, though this embodiment uses an X-ray CT apparatus having a multi-row X-ray detector, an X-ray CT apparatus having a single-row X-ray detector could also provide a similar effect.

1. An X-ray CT apparatus comprising:
X-ray data acquisition means for acquiring X-ray projection data of an X-ray passed through a subject positioned between an X-ray generator and a multi-row X-ray detector which are opposite to each other; and
image reconstruction means for reconstructing image from the projection data acquired by the X-ray data acquisition means;
wherein said image reconstruction means includes means for correcting the X-ray projection data lacking in part or deteriorated in S/N ratio.

2. The X-ray CT apparatus according to claim 1, wherein said image reconstruction means includes means for correcting the X-ray projection data lacking in part or deteriorated in S/N ratio using the projection data of a view with no lack of X-ray projection data.

3. The X-ray CT apparatus according to claim 1, wherein said image reconstructing means includes means for correcting the X-ray projection data lacking in part or deteriorated in S/N ratio using a characteristic parameter of a view with no lack of X-ray projection data.

4. The X-ray CT apparatus according to claim 1, wherein said image reconstructing means includes means for correcting the X-ray projection data lacking in part or deteriorated in S/N ratio using a scout image.

5. The X-ray CT apparatus according to claim 1, wherein said image reconstructing means includes means for correcting the X-ray projection data lacking in part or deteriorated in S/N ratio using a characteristic parameter of a scout image.

6. The X-ray CT apparatus according to claim 3, wherein said characteristic parameters include a profile area.

7. The X-ray CT apparatus according to claim 5, wherein said characteristic parameters include a profile area.

8. The X-ray CT apparatus according to any of claim 1, wherein said image reconstructing means includes means for correcting the X-ray projection data lacking in part or deteriorated in S/N ratio by using a quantity of correction of X-ray projection data collected on the basis of positional information of the channel direction of an X-ray collimator.

9. The X-ray CT apparatus according to any of claim 1, wherein said image reconstructing means includes means for correcting the X-ray projection data lacking in part or deteriorated in S/N ratio by using a quantity of correction of X-ray projection data collected on the basis of positional information of a beam forming X-ray filter.

10. The X-ray CT apparatus according to any of claim 1, wherein said image reconstructing means includes means for correcting the X-ray projection data lacking in part or deteriorated in S/N ratio by adding X-ray projection data using information on the profile area of scout images or the profile area of X-ray projection data of a view with no lack of X-ray projection data so as to obtain constant profile area of the X-ray projection data of each view.

11. The X-ray CT apparatus according to any of claim 1, further including imaging condition setting means to set a region of interest desired to be imaged; and
wherein said image reconstructing means includes means for correcting the X-ray projection data lacking in part or deteriorated in S/N ratio by adding X-ray projection data using information on the profile area of scout images or the profile area of X-ray projection data of a view with no lack of X-ray projection data so as to obtain constant profile area of the X-ray projection data of each view, which position and a profile area of the X-ray data to be added varies according to a position of the region of interest desired to be imaged.

12. The X-ray CT apparatus according to claim 11, wherein said X-ray data acquisition means includes at least one of a channel direction X-ray collimator which tracks in the channel direction the region of interest desired to be imaged while acquisition X-ray projection data and a beam forming X-ray filter.

13. The X-ray CT apparatus according to claim 12, wherein said X-ray data acquisition means includes a controller which feed forward control at least one of the channel direction X-ray collimator and the beam forming X-ray filter in accordance with the channel position and the channel aperture width obtains in advance by calculation for each view or views at constant intervals for an region of interest of a preset region desired to be imaged of the subject.

14. The X-ray CT apparatus according to claim 12, wherein said X-ray data acquisition means includes a controller which feed back control a deviations between a setpoint and a measurement of the position in the channel direction and aperture width in the channel direction according to a measurement of at least one of the channel direction X-ray collimator and the beam forming X-ray filter from the output of the X-ray detector in each view or views at constant intervals.

15. The X-ray CT apparatus according to claim 13, wherein said image reconstructing means includes means for correcting the X-ray projection data lacking in part outside the aperture width in the channel direction or deteriorated in S/N ratio by using information on the profile area of scout images or the profile area of X-ray projection data of a view with no lack of X-ray projection data and adding X-ray projection data so as to obtain constant profile area of the X-ray projection data of each view.

16. The X-ray CT apparatus according to claim 14, wherein said image reconstructing means includes means for correcting the X-ray projection data lacking in part outside the aperture width in the channel direction or deteriorated in S/N ratio by using information on the profile area of scout images or the profile area of X-ray projection data of a view with no lack of X-ray projection data and adding X-ray projection data so as to obtain constant profile area of the X-ray projection data of each view.

17. The X-ray CT radioscopy apparatus comprising:
X-ray data acquisition means for acquiring X-ray projection data of an X-ray passed through a subject positioned between an X-ray generator and a multi-row X-ray detector which are opposite to each other; and
image reconstruction means for reconstructing image from the projection data acquired by the X-ray data acquisition means;
wherein said image reconstruction means includes means for correcting the X-ray projection data lacking in part or deteriorated in S/N ratio.

18. The X-ray CT fluoroscopic apparatus according to claim 17, wherein:
the channel direction X-ray collimator or the beam forming X-ray filter is fixed in the central part or near the central part in the channel direction, and low exposure to radiation is realized by making the central part of the image reconstruction area the region of interest and aligning the region of interest of the subject with the central part of the image reconstruction area.
